(19) United States
(12) Patent Application Publication
(10) Pub. No.: US 2022/0062166 A1
(43) Pub. Date: Mar. 3, 2022

(54) Self-Gelling Solutions for Administration of Therapeutics to the Inner Ear

(71) Applicant: Spiral Therapeutics, Inc., San Francisco, CA (US)

(72) Inventors: Carmen Herrero, Barcelona (ES); Andrew Ayoob, San Francisco, CA (US); Justin Hanes, Baltimore, MD (US); Hugo Peris, San Francisco, MD (US)

(21) Appl. No.: 17/420,899

(22) PCT Filed: Jan. 9, 2020
(86) PCT No.: PCT/US2020/012942

§ 371 Date: Jul. 6, 2021

Related U.S. Application Data
(63) Continuation of application No. 16/243,908, filed on Jan. 9, 2019, now abandoned.

(51) Int. Cl. A61K 9/00; A61P 27/16; A61K 9/06; A61K 31/4985; A61K 47/02; A61K 47/24; A61K 47/34

(52) U.S. Cl. CPC A61K 9/0046; A61P 27/16; A61K 9/0019; A61K 47/34; A61K 31/4985; A61K 47/02; A61K 47/24; A61K 9/06

(57) ABSTRACT

A solution for sustained release of therapeutic, prophylactic and/or diagnostic agent in the inner ear has been developed. The formulation can be injected through a small gauge needle into the inner ear, where it gels to form a sustained release depot for controlled delivery of drug over a few days. In the preferred embodiment, the formulation includes a thermoresponsive sol-gel polymer such as POLOXAMER 407 which forms a stable hydrogel after trans-tympanic injection. As demonstrated by the examples, the hydrogel provides sustained release of an apoptosis inhibitory agent, LPT99, an anti-apoptosis agent that inhibits apoptotic protease activating factor 1 (APAF-1), as well as safety and efficacy in in vitro and in vivo models.

### CROSS-REFERENCE TO RELATED APPLICATIONS

This application claims the benefit of and priority to U.S. Utlity application Ser. No. 16/243,908 filed Jan. 9, 2019, which is hereby incorporated by reference in its entirety.

### FIELD OF THE INVENTION

The invention is in the field of formulations for treatment of inner ear conditions or disease, particularly solutions which form a stable hydrogel at body temperature to provide controlled delivery over a period of days of therapeutic, prophylactic and/or diagnostic agent.

### BACKGROUND OF THE INVENTION

In recent years there has been increasing interest in the treatment of inner ear disorders by local rather than systemic application of drugs, as reviewed by Salt, et al. Drug Discov Today. 2005 Oct. 1; 10(19): 1299-1306. Substances are applied intratympanically, i.e. injected through the tympanic membrane into the middle ear cavity. This procedure is based on the premise that the drug will contact the round window membrane (RWM) of the cochlea, enter the scala tympani (ST) and spread throughout the ear. The target tissues of such treatments may include the sensory hair cells, the afferent nerve fibers and supporting cells of the cochlea (hearing) or vestibular (balance) portions of the inner ear.

The idea of a topical application of medicine to the inner ear is not new. Local anaesthetics and aminoglycosides were applied decades ago to treat inner ear disorders. The present, most widely used form of intratympanic therapy is the injection of gentamicin into the middle ear in patients with Meniere's disease. Gentamicin is toxic to the sensory cells of the balance system and thereby suppresses the vertigo in these patients by partially ablating their vestibular system. There are also an increasing number of clinical reports related to the local application of glucocorticoids for acute hearing loss, Meniere's disease or for tinnitus. Other substances that have been tested in humans include local anaesthetics, neurotransmitters and neurotransmitter antagonists. There is also interest in the use of growth factors, antioxidants, apoptosis inhibitors and antisense-oligonucleotides. Animal experiments have shown promising results using locally applied drugs to provide otoprotection from noise and drug toxicity. One extension of such studies is local viral and non-viral gene transfer for the sustained treatment of inner ear disorders.

Local application of drugs to the inner ear is based on the rationale that despite the lower total amount of drug given, medications applied topically to the RWM can result in higher concentrations in the inner ear fluids than would be the case with systemic application. Potential side effects of systemic treatment and complications from a long lasting, higher dose therapy can be avoided through topical application therapy. Substances applied locally at a low dose can be administered where there are major restrictions or even contraindications associated with systemic application.

Treatment of inner ear conditions is difficult. Most drugs have to be administered constantly as needed since formulations tend to drain out of the treated area. For treatment of the inner ear, this may mean repeated injection or having to use systemic levels to achieve efficacy within the ear.

Currently available formulations that form a solid or semi-solid are formed from suspensions, which may result in a lack of uniform drug distribution and release, with poor pharmackokinetics.

Therefore, it is an object of the invention to provide formulations with beneficial effects that can be administered for sustained local delivery of protective agents over a period of days within the ear, minimizing risk of systemic exposure.

It is another object of the invention to provide formulations with uniformly dissolved therapeutic, prophylactic or diagnostic agents, providing controlled release and pharmacokinetics.

### SUMMARY OF THE INVENTION

A solution for sustained release of therapeutic, prophylactic and/or diagnostic agent in the inner ear has been developed. The formulation can be injected through a small gauge needle into the inner ear, where it gels to form a sustained release stable hydrogel depot for controlled delivery of agent over a few days. The hydrogel provides sustained release of agent for a period of between at least three to fifteen days in the ear.

In a preferred embodiment, the hydrogel forming excipient is POLOXAMER® 407. The hydrogel forming polymer constitutes between 10% and 30% by weight of the polymer solution, which may contain other excipients and polymers, with the most preferred amount of a polymer such as POLOXAMER® 407 constituting about 15% w/w of the formulation. This is a solution, not a suspension, which is extremely stable at room temperature for a period of at least three months.

Prior to introducing the agent, the phase-transition hydrogel forming polymer such as POLOXAMER® 407 is formulated as a liquid product including an amount of POLOXAMER® 407 that at body temperature forms a hydrogel providing sustained release of agent. The agent(s) is added to the formulation to form a homogeneous solution without causing gelation. The formulation has a viscosity suitable for injection through a 23-G needle, typically through the tympanic membrane into the tympanic cavity. The formulation may further include sodium chloride, water, antioxidants, antimicrobials, detergents, solubilizing agents, crystallization inhibitors, viscosity modifiers, chelators, and buffers including, but not limited to, hydrogen phosphate di-sodium dodecahydrate and dihydrogen sodium phosphate dihydrate.

### BRIEF DESCRIPTION OF THE DRAWINGS

FIG. 1 is a cross-sectional view of the anatomy of the middle and inner ear.

FIG. 2A-2B are graphs of temperature-induced gelation of LPT99-H1 in a POLOXAMER solution, viscosity (cP) versus temperature (° C.). FIG. 2A is over a range of 5-25° C.; FIG. 2B is over a range of 5-37° C.

FIG. 3A-3B are graphs of LPT99 release (FIG. 3A, μg/g; FIG. 3B, %) over days in situ.

FIG. 4 is a graph of LPT99 concentration (ng/g) over days since intratympanic injection of 100 and 478 μM LPT99.

FIG. 5A-5B are graphs of LPT99 concentrations within cochlea harvested and rinsed at several timepoints after intratympanic injection of drug product (5A, left ear; 5B, right ear). Drug concentration in cochlear homogenates is expressed as nanograms LPT99 per gram of cochlear homogenate.

Treatments were:
Vehicle cisplatin+Vehicle SPT991 (n=10)
Cisplatin+Vehicle SPT991 (n=9)
Cisplatin+SPT991 63 μg/mL (n=10)
Cisplatin+SPT991 300 μg/mL (n=10)
Cisplatin+LPT-99-CD (n=10)

FIGS. 6A, 6B, and 6C are graphs of ABR threshold (dB) versus frequency (kHz) 24 hrs (FIG. 6A), 10 days (FIG. 6B), and 21 days (FIG. 6C) after administration, for:
[symbol] CONTROL (n=11, 2 ears)
[symbol] CONTROL+VEHICLE (N=11, 2 ears)
[symbol] TRAUMA (n=15, 2 ears)
[symbol] TRAUMA+LPT99 (n=15, 2 ears)

The results demonstrate that noise exposure induces an increase in ABR Threshold shift in non-treated groups. Noise-induced ABR Threshold Shift is present at 1, 10 and 21 days in LPT99-treated groups, ABR Threshold shift is back at basal levels at 10 and 21 days.

### DETAILED DESCRIPTION OF THE INVENTION

### I. Definitions

“Active agent” and “active pharmaceutical ingredient” are used interchangeably and refer to a physiologically or pharmacologically active substance that acts locally and/or systemically in the body. An active agent is a substance that is administered to a patient for the treatment (e.g., apoptotic inhibitory agent), prevention (e.g. agent), or diagnosis (e.g. agent) of a disease or disorder.

The term “adenosine receptor 3” or “A3” or “ADORA3” is a purinergic G-coupled receptor involved in a variety of intracellular signaling pathways.

The term “ADME” is an abbreviation in pharmacokinetics and pharmacology for “absorption, distribution, metabolism, and excretion”, and describes the disposition of a pharmaceutical compound within an organism. The four criteria all influence the drug levels and kinetics of drug exposure to the tissues and hence influence the performance and pharmacological activity of the compound as a drug.

The term “Apaf-1” or “apoptotic protease activating factor-1” is a cytoplasmic protein that forms one of the central hubs in the apoptosis regulatory network. Upon binding cytochrome c and dATP, this protein forms an oligomeric apoptosome which binds and cleaves Procaspase 9 protein, releasing its mature, activated form.

The term “apoptosis” means is a process of programmed cell death that occurs in multicellular organisms. Biochemical events lead to characteristic cell changes (morphology) and death. Apoptosis is a highly regulated and controlled process that confers advantages during an organism's lifecycle.

The term “AUC” or “area under the curve” in the field of pharmacokinetics, the area under—the curve (AUC) is the definite integral in a plot of drug concentration in blood plasma versus time. In practice, the drug concentration is measured at certain discrete points in time and the trapezoidal rule is used to estimate AUC.

The term “auditory brainstem response” or “ABR” refers to an auditory evoked potential extracted from ongoing electrical activity in the brain and recorded via electrodes placed on, for example, the scalp. ABR is considered an exogenous response because it is dependent on external factors.

The term “blood labyrinth barrier” or “BLB” refers to the barrier between the vasculature and the inner ear fluids, either endolymph or perilymph. The BLB is critical for the maintenance of the inner ear fluid ionic homeostasis.

The term “BLLQ” is an abbreviation for “below the lower limit of quantification” and is defines as below the lowest standard on the calibration curve.

The term “cholecystokinin receptor 1” or “CCK1” is a G-protein coupled receptor that bines sulfated members of the cholecystokinin family of peptide hormones.

The term “Cₘₐₓ” refers to the maximum (or peak) concentration that a drug achieves in a specified compartment or test area of the body after the dug has been administered and before the administration of a second dose. It is a standard measurement in pharmacokinetics and is the opposite of Cmin.

The term “Cmin” refers to the minimum (or trough) concentration that a drug achieves after dosing.

The term “Cytc” or “cyctochrome c” refers to a small hemeprotein found loosely associated with the inner membrane of the mitochondrion. It has an intermediate role in apoptosis in activating caspase 9 via the apoptosome.

The term “cytocochleogram” refers to a graphic representation of the anatomical state of the hair cells along the complete width and length of the organ of Corti.

The abbreviation “DDI” refers to drug-drug interaction.

The term “DFNB29” or “deafness, autosomal recessive 29” refers to a chromosomal locus where recessive mutations of CLDN14 encoding claudin 14 results in human hereditary deafness. The DFNB29 phenotype is characterized by pre-lingual, bi-lateral, sensorineural hearing loss.

The term “drug absorption” or “absorption” refers, preferably, to the process of movement of the active agent from the localized site of administration, by way of example only, the round window niche of the cochlea, and across a barrier (the round window membrane, as described below) into the auris interna or inner ear structures. The terms “co-administration”, as used herein, are meant to encompass, preferably, administration of the otic agent to a single patient, and are intended to include prevention regimens in which the otic agents are administered by the same or different route of administration or at the same or different time.

The terms “effective amount” or “therapeutically effective amount,” as used herein, refer to a sufficient amount, preferably, of the otic agent being administered that would be expected to relieve to some extent one or more of the symptoms of the disease or condition being prevented, i.e. , a quantity necessary to render the desired apoptotic inhibitory result. The term “therapeutically effective amount” includes, for example, an “effective amount” of an otic agent to achieve a desired pharmacologic effect or apoptotic inhibitory improvement without undue adverse side effects. It is understood that “an effective amount” or “a therapeutically effective amount” varies, in some implementations, from subject to subject, due to variation in metabolism of the compound administered, age, weight, general condition of the subject, the condition being prevented, the severity of the condition being prevented, and the judgment of the prescribing physician. It is also understood that “an effective amount” in an extended-release dosing format may differ from “an effective amount” in an immediate-release dosing format based upon pharmacokinetic and pharmacodynamic considerations.

The term “enhance” or “enhancing,” refers to an increase or prolongation of either the potency or duration of a desired effect, preferably, of the otic agent, or a diminution of any adverse symptomatology. For example, in reference to enhancing the effect of the otic agents disclosed herein, the term “enhancing” refers to the ability to increase or prolong, either in potency or duration, the effect of other apoptotic inhibitory agents that are used in combination with the otic agents disclosed herein. An “enhancing-effective amount,” as used herein, refers to an amount of an otic agent or other apoptotic inhibitory agent that is adequate to enhance the effect of another apoptotic inhibitory agent or otic agent in a desired system. When used in a patient, amounts effective for this use will depend on the severity and course of the disease, disorder or condition, previous therapy, the patient's health status and response to the drugs, and the judgment of the preventing physician.

The term “GLP” refers to “good laboratory practice” and is a set of principles intended to assure the quality and integrity of non-clinical laboratory studies.

The term “hERG” refers to human ether-a-go-go-related gene that codes for a protein that is the alpha subunit of a potassium ion channel

The term “IC₅₀” refers to the concentration of an inhibitor where the response (or binding) is reduced by half.

The terms “inhibit” and “reduce” mean to reduce or decrease in activity or expression. The terms also include preventing, slowing, or reversing the development of a condition, for example, ototoxcity, or advancement of a condition in a patient necessitating prevention. This can be a complete inhibition or reduction of activity or expression, or a partial inhibition or reduction. Inhibition or reduction can be compared to a control or to a standard level. The term “MRSD” or “maximum recommended starting dose” refers to the highest amount of an agent that can be given safely and without complication while maintaining its efficacy.

The term “MTD” or “maximum tolerated dose” refers to the highest dose of a drug or prevention that does not cause unacceptable side effects.

The term “NOAEL” refers to “no observed adverse effect level” and is an important part of the non-clinical risk assessment.

The term “ototoxicity” means the property of being toxic to the ear, specifically the cochlea, including the cochlear sensory hair cells, or auditory nerve and sometimes the vestibular system, for example, as a side effect of a drug. The effects of ototoxicity can be reversible and temporary, or irreversible and permanent. There are many well-known ototoxic drugs used in clinical situations, and they are prescribed, despite the risk of hearing disorders, for treatment of very serious health conditions such as aggressive cancers or bacterial infections. Ototoxic drugs include antibiotics such as gentamicin, loop diuretics such as furosemide and platinum-based chemotherapy agents such as cisplatin. A number of nonsteroidal anti-inflammatory drugs (NSAIDS) have also been shown to be ototoxic. This can result in sensorineural hearing loss, dysequilibrium, or both. Some environmental and occupational chemicals have also been shown to affect the auditory system.

The term “pharmaceutically acceptable salts” means those salts which conserve the efficiency and the biological properties of the free bases or free acids.

The term “auris-acceptable penetration enhancer” or “penetration enhancer” refers to an agent that reduces barrier resistance (e.g., barrier resistance of the round window membrane).

The term “pharmacodynamics” refers to the factors that determine the biologic response observed relative to the concentration of drug at the desired site, such as within the auris media and/or auris interna.

The term “pharmacokinetics” refers to the movement of the drug factors that determine the attainment and maintenance of the appropriate concentration of drug at the desired site, such as within the auris media and/or auris interna.

The term “platinum-based antineoplastic drugs” or “platins” are chemotherapeutic agents such as cisplatin, oxaliplatin, and carboplatin, used to kill cancerous cells. They are coordination complexes of platinum. These drugs are used to treat almost half of people receiving chemotherapy for cancer.

The term “prophylactically effective amount or dose” refers to an amount of a composition administered to a patient susceptible to or otherwise at risk of a particular disease, disorder or condition, for example, ototoxicity. For example, the apoptotic inhibitory formulation may be administered to an individual prior to chemotherapy to prevent hearing loss by the subsequently administered chemotherapeutic agent.

The term “room temperature” refers to a temperature between about 15° C. and less than about 27° C., preferably 25° C.

The term “body temperature” refers to a temperature between about 36.5° C. and about 37.5° C., preferably 37° C.

The term “ROS” or “reactive oxygen species” are chemically reactive chemical species containing oxygen.

“Small molecule” generally refers to an organic molecule that is less than about 2000 g/mol in molecular weight, less than about 1500 g/mol, less than about 1000 g/mol, less than about 800 g/mol, or less than about 500 g/mol. In some forms, small molecules are non-polymeric and/or non-oligomeric.

“Steady state,” refers to when the amount of drug administered, preferably, to the auris media and/or auris interna is equal to the amount of drug eliminated within one dosing interval resulting in a plateau or constant levels of drug exposure within the targeted structure.

“Stable” as used herein refers to chemical and physical stability over a time period under defined conditions. Physical stability refers to a high percentage or all of what was originally dissolved remaining in solution. In a preferred embodiment this value is greater than 60, 70, 80, 90, or 100% remaining dissolved at room temperature (approximately 15-25° C., most preferably 25° C.).

“Sustained release” as used herein refers to release of a substance over an extended period of time in contrast to a bolus type administration in which the entire amount of the substance is made biologically available at one time.

The term “Tₘₐₓ” refers to the time it takes a drug or other substance to reach the maximum concentration Cₘₐₓ.

The term “transtympanic administration” refers to the administration of a therapeutic, or agent via the tympanic cavity, preferably via a hypodermal needle that accesses the tympanic cavity (middle ear) by penetrating the tympanic membrane (eardrum).

The terms “prevent,” “preventing” or “prevention,” as used herein, include alleviating, abating or ameliorating a disease or condition, for example ototoxicity, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or controlling or stopping the symptoms of the disease or condition.

### II. Controlled Release Apoptosis Inhibitory Compositions

Auris or otic compositions have been developed for extended release, either continuously or in a pulsatile manner, or variants of both, of therapeutic, prophylactic and/or diagnostic agent(s) within the ear. The extended release otic composition increases the area under the curve (AUC) of the agent being delivered in otic fluids (e.g., endolymph and/or perilymph) by about 30%, about 40%, about 50%, about 60%, about 70%, about 80% or about 90% compared to a composition that is not a extended release otic composition. The extended release compositions may also decrease the Cₘₐₓ in otic fluids (e.g., endolymph and/or perilymph) by about 40%, about 30%, about 20%, or about 10%, compared to a composition that is not an extended release otic composition. This reduces the ratio of Cₘₐₓ to Cₘᵢₙ compared to a composition that is not an extended release otic composition. In certain implementations, the ratio of Cₘₐₓ to Cₘᵢₙ is 10:1, 9:1, 8:1, 7:1, 6:1, 5:1, 4:1, 3:1, 2:1 or 1:1. The length of time that the concentration of an otic agent is above Cₘᵢₙ by about 30%, about 40%, about 50%, about 60%, about 70%, about 80% or about 90% compared to a composition that is not a extended release otic composition. In certain instances, the extended release compositions delay the time to Cₘₐₓ, and/or prolongs the time the concentration of the drug will stay above the Cₘᵢₙ. In some forms, auris compositions prolong the residence time of a drug in the inner ear. In the preferred embodiment, once the concentration in the endolymph or perilymph of a drug reaches steady state, the concentration of the drug in the endolymph or perilymph stays at or about the apoptotic inhibitory dose for an extended period of time (e.g., one day, 2 days, 3 days, 4 days, 5 days, 6 days, or 1 week).

The compositions have at least three components: therapeutic, prophylactic and/or diagnostic agent; gel forming polymer; and other excipients, which together form an extended release composition to be administered into the ear.

A. Therapeutic, Prophylactic and Diagnostic Agents

Many therapeutic, prophylactic and diagnostic agents are administered to the ear. These include antinfectives, immunomodulators, anti-inflammatories, local anesthetics, analgesics, aminoglycosides, compounds such as gentamicin for treatment of Menière's disease, neurotransmitters and neurotransmitter antagonists, growth factors, antioxidants, apoptosis inhibitors and a variety of gene therapy nucleic acids including antisense oligonucleotides, si-RNA, miRNA and others for the sustained treatment of inner ear disorders.

Diagnostic agents include dyes, fluorophores, and other agents detectable by ultrasound, MRI, or x-ray.

B. Controlled Release Excipient

The composition, formulated for otic delivery, is in the form of a solution that effects a transition from a liquid state at room temperature to a hydrogel at body temperature. This is important so that the formulation can be injected into the inner ear, preferably using a small diameter needle (23G or smaller), where it then solidifies, typically through a sol-gel transition effected by the increased temperature of the body relative to the temperature at which the formulation was prepared and/or stored.

The compositions can contain additional components such as pH buffers, tonicity agents, mucoadhesive agents, stabilizing agents, preservatives, carriers, viscosity enhancing agents, and penetration enhancers.

The pH of the composition is preferably between 6.8 and 7.7, most preferably 7.2. The composition preferably has an osmolality of about 280 mOsmol/kg.

### Thermosensitive Hydrogel Forming Polymers

Hydrogels are formed of networks of physically or chemically crosslinked polymers imbibed with aqueous media such as water or biological fluids. Chemical crosslinks (covalent bonds) or physical junctions (e.g. hydrophobic associations, crystallite formation, chain entanglements) provide the hydrogels' three-dimensional structure. Hydrogels have been a topic of extensive research in the past decades and their properties, such as their high water content and the possible control over the swelling kinetics. In situ forming hydrogels provide a means for wherein a polymer solution is prepared and allowed to gel in situ, after photopolymerization, chemical crosslinking, ionic crosslinking or in response to an environmental stimulus such as temperature, pH or ionic strength of the surrounding medium. Hydrogels that are sensitive to thermal stimuli are useful as temperature is the sole stimulus for their gelation with no other requirement for chemical or environmental treatment and can be thus produced e.g. upon injection to the body, when temperature is increased from ambient to physiological.

The phenomenon of transition from a solution to a gel is commonly referred to as sol-gel transition. Some hydrogels exhibit a phase transition from a liquid solution to a solid hydrogel above a certain temperature. This threshold is defined as the lower critical solution temperature (LCST). Below the LCST, the polymers exist as single chains or are associated in unpacked micelles. Above the LCST, they become increasingly hydrophobic and insoluble, leading to gel formation. Hydrogels that are formed upon cooling of a polymer solution have an upper critical solution temperature (UCST). The sol-gel transition of thermosensitive hydrogels can be experimentally verified by a number of techniques such as the vial inversion method, spectroscopy, differential scanning calorimetry (DSC) and rheology.

In some instances, intra-tympanic injection of cold compositions (e.g., a composition with temperatures of <20° C.) causes a density gradient in the inner ear fluids that induces vertigo, a phenomenon called nystagmus, in individuals undergoing prevention for inner ear disorders. Preferably, the compositions are designed to be liquids that are administered at or near room temperature and do not cause vertigo or other discomfort when administered to an individual or patient.

Some natural polymers can transition form a liquid to a solid state based on temperature, such as some of the modified cyclodextrins, but these are not preferred.

“Synthetic polymers” that transition from a liquid to solid state refers to polymers that are auris-acceptable such as copolymers of ethylene oxide and propylene oxide, (e.g., poloxamers (PLURONICS® (BASF)) such as POLOXAMER® 407 and POLOXAMER® 188). Preferred polymers are synthetic polymers such as N-isopropylacrylamide (NiPAAM) polymers, poly(ethylene oxide)-b-poly(propylene oxide)-b-poly(ethylene oxide) (PEO-PPO-PEO) as well as poly(ethylene glycol) (PEG)-biodegradable polyester copolymers. POLOXAMERS® include PLURONICS® F68, F88, F108, and F127 which are block copolymers of ethylene oxide and propylene oxide); and POLOXAMINES® (e.g., TETRONIC® 908, also known as POLOXAMINE® 908, which is a tetrafunctional block copolymer derived from sequential addition of propylene oxide and ethylene oxide to ethylenediamine (BASF Corporation, Parsippany, N.J.)),

Preferred formulations contain a POLOXAMER®, triblock copolymers of poly(ethylene oxide) (PEO) and poly(propylene oxide) (PPO) available in different molecular weights and PPO/PEO ratios. The hydrogel provides sustained release of the apoptosis inhibitory agent for a period of at least 3-15 days in the ear. In a preferred embodiment, the hydrogel forming excipient is POLOXAMER® 407.

POLOXAMER® 407 (F-127) is a nonionic polymer composed of polyoxyethylene-polyoxypropylene copolymers. Other commonly used poloxamers include 188 (F-68 grade), 237 (F-87 grade), 338 (F-108 grade). Aqueous solutions of poloxamers are stable in the presence of acids, alkalis, and metal ions. PF-127 is a commercially available poly(oxyethylene)-poly(oxypropylene) triblock copolymer of general formula E106 P70 E106, with an average molar mass of 13,000 Da. In the general formula shown above, E and P denote poly(oxyethylene) and poly(oxypropylene), respectively; and the integers 106 and 70 denote the degree of polymerization of the polymers. PF-127 contains approximately 70% ethylene oxide, which provides for its hydrophilicity.

The amount of polymer, such as the thermoreversible polymer, may be about 10%, about 15%, about 20%, about 25%, about 30%, or about 35% of the total weight of the composition. In some forms, the amount of thermoreversible polymer is about 14%, about 15%, about 16%, about 17%, about 18%, about 19%, about 20%, about 21%, about 22%, about 23%, about 24% or about 25% of the total weight of the composition. In a particular implementation, the polymer is POLOXAMER® 407 at a concentration of 17.3% (w/v).

In some forms, synthetic polymers are included to enhance physical stability or for other purposes. Some other synthetic polymers include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40; polysorbates such as polyethylene glycol sorbitan monostearate and polyethylene glycol sorbitan monooleate; triacetin; D-a-tocopheryl polyethylene glycol succinate (vitamin E TPGS); phospholipids; lecithins; phosphatidyl cholines (c8-c18); phosphatidylethanolamines (c8-c18); phosphatidylglycerols (c8-c18); bile salts; glyceryl monostearate; polyoxyethylene fatty acid glycerides; vegetable oils such as polyoxyethylene (60) hydrogenated castor oil; and polyoxyethylene alkylethers and alkylphenyl ethers such as octoxynol 10, octoxynol 40; or a combination thereof.

In some forms, the excipient enhances solubility of the apoptosis inhibitory agent between about, 30-fold, 100-fold, 300-fold, or 1000-fold, compared to a corresponding composition lacking the synthetic polymer or to water.

### Other Additives and Excipients

Other materials can be incorporated into the hydrogel forming material. Representative materials include diluents, buffers, dispersing agents or viscosity modifying agents, solubilizers, stabilizers, and osmolarity modifying agents.

The term “diluent” refers to chemical compounds that are used to dilute, preferably, the otic agent prior to delivery, and which are compatible, preferably, with the auris media and/or auris interna.

The term “dispersing agents,” and/or “viscosity modulating agents” and/or “thickening agents” refer to materials that enhance dispersion of particulate matter in a solution or modify the viscosity of a solution or suspension. Examples of dispersing agents/materials include, but are not limited to, hydrophilic polymers, electrolytes, TWEEN® 60 or TWEEN® 80, PEG, polyvinylpyrrolidone (PVP; also known as povidone and commercially known as Kollidon®, and PLASDONE®), and the carbohydrate-based dispersing agents such as, for example, modified celluloses such as hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose, carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), and polyethylene glycol, having a molecular weight of about 300 to about 6000, or about 3350 to about 4000, or about 7000 to about 5400. In some embodiments, the amount of thickening agent is about 1%, 5%, about 10%, or about 15% of the total weight of the composition. In some instances, dispersants improve composition stability by inhibiting drug crystallization.

The compositions have a suitable viscosity for injection through a 23-G needle or a needle of a higher gauge. At elevated temperatures (above 26° C.), the viscosity increases (due to the sol-gel transition) to above 100,000 cP. At 14.73 w/w P407, the viscosity is about 100 cP at temperatures below 20° C.

The term “solubilizer” refers to auris-acceptable compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins and other cyclodextrins, ethanol, n-butanol, isopropyl alcohol, cholesterol, bile salts, polyethylene glycol 200-600, glycofurol, TRANSCUTOL®, propylene glycol, and dimethyl isosorbide, ethanol, and other organic solvents. Preferred solvents are propylene glycol, PEG300, ethanol, and cyclodextrins.

The term “stabilizer” refers to compounds such as antioxidants, buffers, acids, and preservatives that are compatible, preferably, with the environment of the auris media and/or auris interna. Stabilizers include agents that improve the compatibility of excipients with a container, or a delivery system, including a syringe or a glass bottle, improve the stability of a component of the composition, or improve composition stability.

Tonicity and pH adjusting agents may be added. In general, the endolymph has a higher osmolality than the perilymph. For example, the endolymph has an osmolality of about 304 mOsm/kg H₂O, while the perilymph has an osmolality of about 294 mOsm/kg H₂O. In some forms, the otic or auris compositions are formulated to provide an osmolality between about 100 mOsm/kg and about 500 mOsm/kg, between about 200 mOsm/kg and about 400 mOsm/kg, between about 240 mOsm/kg and about between 350 mOsm/kg, between about 250 mOsm/kg and about 350 mOsm/kg, between about 270 mOsm/kg and about 320 mOsm/kg, or between about 280 mOsm/kg and about 320 mOsm/kg. In some forms, the compositions have an osmolality of about 280 mOsm/kg. In some forms, the compositions have an osmolarity between about 100 mOsm/L and about 500 mOsm/L, between about 200 mOsm/L and about 400 mOsm/L, between about 240 mOsm/L and about between 350 mOsm/L, between about 250 mOsm/L and about 350 mOsm/L, between about 270 mOsm/L and about 320 mOsm/L, or between about 280 mOsm/L and about 320 mOsm/L. In some forms, the osmolarity of the composition is designed to be isotonic with the targeted otic structure (e.g., endolymph, perilymph or the like).

Osmolarity/osmolality is adjusted, for example, by the use of appropriate salt concentrations (e.g., concentration of potassium salts) or the use of tonicity agents, which renders the compositions endolymph-compatible and/or perilymph-compatible (i.e., isotonic with the endolymph and/or perilymph. In some instances, the compositions, preferably endolymph-compatible and/or perilymph-compatible compositions, cause minimal disturbance to the environment of the inner ear and cause minimum discomfort (e.g., vertigo and/or nausea) to a mammal upon administration.

In some forms, the composition is isotonic with the perilymph. Isotonic compositions are provided by the addition of a tonicity agent. Suitable tonicity agents include, but are not limited to, any pharmaceutically acceptable sugar, salt or any combinations or mixtures thereof, such as, but not limited to dextrose, glycerin, mannitol, sorbitol, sodium chloride, and other electrolytes. Sodium chloride or other tonicity agents are optionally used to adjust tonicity, if necessary. Representative salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate. A preferred salt is sodium chloride.

The formulations typically include one or more pH-adjusting agents or buffering agents. Suitable pH adjusting agents or buffers include acetate, bicarbonate, ammonium chloride, citrate, phosphate, pharmaceutically acceptable salts thereof and combinations or mixtures thereof. Suitable water-soluble buffering agents are alkali or alkaline earth metal carbonates, phosphates, bicarbonates, citrates, borates, acetates, succinates and the like, such as sodium phosphate, citrate, borate, acetate, bicarbonate, carbonate and tromethamine (TRIS).

In some forms, the compositions include a mucoadhesive. Preferably, the mucoadhesive facilitates adhesion to a portion of the ear, such as the external mucous layer of the round window membrane. Mucoadhesive agents include, but are not limited to, carbomers, such as CARBOPOL® 934P, polyvinylpyrrolidone polymer (PVP); a water-swellable, but water-insoluble, fibrous, cross-linked carboxy-functional polymer; a crosslinked poly(acrylic acid) (e.g. CARBOPOL® 947P); a carbomer homopolymer; a carbomer copolymer; a hydrophilic polysaccharide gum; maitodextrin; a cross-linked alginate gum gel, hydroxypropyl methylcellulose, and a water-dispersible polycarboxylated vinyl polymer. Mucoadhesive agents are described in U.S. Pat. No. 8,828,980 to Lichter, et al.

Examples of surfactants include, but are not limited to, sodium lauryl sulfate, sodium decussate, TWEEN® 60 (polyethylene glycol sorbitan monostearate) or TWEEN®80 (polyethylene glycol sorbitan monooleate), triacetin, D-α-tocopheryl polyethylene glycol succinate (vitamin E TPGS), phospholipids, lecithins, phosphatidyl cholines (c8-c18), phosphatidylethanolamines (c8-c18), phosphatidylglycerols (c8-c18), sorbitan monooleate, polyoxyethylene sorbitan monooleate, polysorbates, bile salts, glyceryl monostearate,

The compositions may include penetration enhancers that allow for delivery of the apoptosis inhibitory agents across a barrier, such as the oval window or the round window of the ear. Preferably, the penetration enhancers are auris-compatible. Penetration enhancers include sodium lauryl sulfate, sodium octyl sulfate, sodium dodecyl sulfate, ocytl-trimethyl-ammonium bromine, dodecyl-trimethyl ammonium bromide, sodium laurate, polyoxyethylene-20-cetyl ether, laureth-9, sodium dodecylsulfate, dioctyl sodium sulfosuccinate, polyoxyethylene-9-lauryl ether (PLE), TWEEN® 20, TWEEN® 80, nonylphenoxypolyethylene (NTP-POE), polysorbates, bile salts, fatty acids and derivatives . chelating agents (such as EDTA, citric acid, and salicylates, sulfoxides (such as dimethyl sulfoxide (DMSO) and decylmethyl sulfoxide), and alcohols (such as ethanol, isopropanol, glycerol, and propanediol.

In some forms, the compositions include a preservative. Suitable preservatives include, but are not limited to, benzoic acid, acid, boric acid, p-hydroxybenzoates, alcohols, quaternary compounds, stabilized chlorine dioxide, mercurials, such as merfen and thiomersal, or a combination thereof. Preservatives are described in U.S. Pat. No. 8,828,980 to Lichter, et al.

### C. Concentration, pH, Tonicity of Agent in Excipient

In the preferred formulations, the formulations contain an effective amount of therapeutic, prophylactic and/or diagnostic agent for the desired release period based on the volume of solution to be injected into the ear, and between about 1 μg/mL % w/w and about 10 mg/mL or alternatively 2 mg/mL % w/w, most preferably about 15% w/w, of the polymer such as a poly(ethylene oxide)-poly(propylene oxide) triblock copolymer having the general formula A-B-A or B-A-B, where A is poly(ethylene oxide) and B is poly(propylene oxide). The composition is in the form of a solution that effects a transition from a liquid state at room temperature to a gel state (e.g. hydrogel) at body temperature.

The resulting hydrogel provides sustained release of the therapeutic agent for a period of at least about one day and 30 days, at least five days and 25 days, at least 10 days and 20 days, one day, two days, three days, four days, five days, six days, seven days, 10 days, 15 days, 20 days or 30 days, preferably at least 15 days. The same agent or different agents can be incorporated into the composition for use in single therapy or combination therapy regimens, respectively.

The compositions are formulated to provide a therapeutically effective amount of an agent such as an apoptosis inhibitor across the round window membrane into the cochlea. In the preferred embodiment, the composition contains the therapeutic, prophylactic and/or diagnostic agent or pharmaceutically acceptable prodrug or salt thereof; between about 10% and about 30% by weight of a poly(ethylene oxide)-poly(propylene oxide) triblock copolymer of general A-B-A or B-A-B, where A is poly(ethylene oxide) and B is poly(propylene oxide).

The pH of the composition is between 6 and 8, between 6 and 7.6, more preferably between 6.8 and 7.5, and most preferably 7.2.

The composition can be prepared and stored in vials, syringes, capsules, ampules, or pouches prior to administration. The composition may be packaged in a single-dose that is administered trans-tympanically into the middle ear. Formulations may be lyophilized, micronized, pelleted, or in a solution or suspension. Optionally, the components of the composition are provided in kits that contain instructions to formulate the composition by adding diluent to excipient and/or agent.

### III. Methods of Making

The composition is prepared by mixing an effective amount of therapeutic, prophylactic and/or diagnostic agent in a gel forming solution.

Since the polymer systems of the thermoreversible gel dissolve more completely at reduced temperatures, the preferred methods of solubilization are to add the required amount of polymer to the amount of water to be used. Generally, after wetting the polymer by shaking, the mixture is capped and placed in a cold chamber or in a thermostatic container at between about 0° C. and 10° C. in order to dissolve the polymer. The mixture can be stirred or shaken to bring about a more rapid dissolution of the polymer. Cosolvents can be used to enhance drug solubility; however, some drugs are insoluble. These can often be suspended in the polymer vehicle with the aid of suitable suspending or viscosity enhancing agents.

The agent and various excipients such as buffers, salts, and preservatives can subsequently be added to the polymer-containing gel and dissolved. In some forms the agent is suspended if it is insoluble in water. If needed, the pH can be modulated by the addition of appropriate buffering agents. Preferably, a phosphate buffer is prepared and sterile filtered, and the synthetic polymer is slowly added to cold buffer with stirring, and refrigerated overnight.

### IV. Methods of Using

The formulations are administered to the inner ear of a subject in need thereof. Typically, the subject to be treated is an adult or pediatric human undergoing treatments that can cause hearing loss, such as chemotherapy, hearing loss due to aging, hearing loss due to repeated exposure to loud noises, and other disorders damaging the cilia in the inner ear such as autoimmune disorders, infection, excess fluid or pressure.

Preferred methods of administration of the composition are localized administrations by trans-tympanic injection of the formulation as a solution (i.e., at room temperature or lower). Such administration routes and appropriate compositions are generally known to those of skill in the art. After administration, the composition effects a transition from a liquid state at room temperature to a gel state at body temperature. Preferably, the gel state provides sustained release of the apoptosis inhibitory agent for a period of least about one day and 30 days, at least five days and 25 days, at least 10 days and 20 days, one day, two days, three days, four days, five days, six days, seven days, 10 days, 15 days, 20 days or 30 days, preferably at least 15 days.

In the preferred embodiment, the compositions are administered on or near the round window membrane via trans-tympanic injection. The composition may also be administered on or near the round window or the crista fenestrae cochleae through entry via a post-auricular incision and surgical manipulation into or near the round window or the crista fenestrae cochleae area. Preferably administration is made using a syringe and small gauge needle, 23G to 30G or smaller, wherein the needle is inserted through the tympanic membrane and guided to the area of the round window or crista fenestrae cochleae. The composition is then deposited on or near the round window or crista fenestrae cochleae. In other embodiments, the composition is administered via microcathethers implanted into the subject, using a drug delivery device such as a micropump, a microinjection device, or a microreservoir implanted within the inner ear for long term prevention of hearing loss.

The formulation can also be administered into the tympanic cavity or applied on the tympanic membrane or onto or in the auditory canal by injection, direct instillation or perfusion of the inner ear compartments, or in surgical procedures including, cochleostomy, labyrinthotomy, mastoidectomy, stapedectomy, or endolymphatic sacculotomy.

The compositions can be administered in a single dose or in multiple doses. Certain factors may influence the dosage required to effectively treat or prevent a disorder, including, but not limited to, the severity of the disease or disorder, previous preventions, the general health and/or age of the subject, and other diseases present. It will also be appreciated that the effective dosage of the composition used for prevention may increase or decrease over the course of a particular prevention. Changes in dosage may result and become apparent from the results of assays.

The present invention will be further understood by reference to the following non-limiting examples.

### EXAMPLES

The data presented in the non-limiting examples below show the efficacy of an apoptosis inhibitor, specifically the Apaf1 inhibitor LPT99, in the prevention and/or prevention of ototoxicity such as, but not limited to, ototoxicity caused by platinum-based chemotherapeutic agents.

### Example 1: Preparation of hydrogel for loading 2-(4-(2,4-dichlorophenethyl)-3,6-dioxo-1-(2-(thiophen-2-yl)ethyl)piperazin-2-yl)-N-(2-(5-methoxy-1H-indol-3-yl)ethyl)acetamide (LPT99)

In vitro experiments with LPT99 demonstrated it's specificity for Apaf1, resulting in inhibition of apoptotic protease activating factor 1 (Apaf1). In a cellular model of CisPt-induced apoptosis, LPT99-treated cells showed a decreased release of cyt c from mitochondria, reduced caspase-3 activation, and an improved cell viability, evidence of the cytoprotective effect of LPT99 (Cervantes, et al., IEB Symposium, Montpellier, Abstract P77, “Inhibition of APAF-1 with LPT99 prevents cisplatin-induced apoptosis in HEI-OC1 auditory cells”, Sep. 18, 2016; Maurillo-Cuesta, et al., IEB Symposium, Montpellier, Abstract P78, Inhibition of Apaf1 with LPT99 prevents cisplatin-induced hearing loss, Sep. 18, 2016).

These studies showed that the compound LPT99 could be effective in preventing hearing loss due to exposure to cisplatin in vitro in cell culture, if a formulation could be developed for administration in a single injection which would provide protection during the entire course of treatment of the patient.

### Materials and Methods

In order to effectively deliver LPT99 to the cochlea, an otic extended release composition, specifically, a hydrogel composition for loading LPT99, which becomes a solution after loading LPT99, was developed which was suitable for injection into the inner ear, where it forms a sustained release hydrogel.

For the preparation of buffer, the reagents were weighed one by one on a precision balance inside the laminar flow cabinet. Table 1 below details the composition of the buffer in units of g/L.

TABLE 1
Phosphate buffer composition

| Composition | (g/L) |
|---|---|
| Hydrogen phosphate di-sodium dodecahydrate | 0.6 |
| Dihydrogen sodium phosphate dihydrate | 0.05 |
| Sodium chloride | 0.4 |
| Water for Injection (WFI) | c.s.p. |

First, around 150 ml of WFI was added to a 250 mL beaker and kept under magnetic stirring. The reagents were then added as follows:

0.05 g of Dihydrogen sodium phosphate dihydrate are weighed into an aluminum weighing pan (WPAL-072-100) and added to the WFI being stirred. To ensure that everything was added, the rest of the reagent that can remain on the weighing pan is washed with WFI.

The same procedure was followed to add hydrogen phosphate di-sodium dodecahydrate and Sodium chloride. After all reagents were weighed and added, the buffer was kept under magnetic stirring in the beaker for 15 minutes. After this time, the solution was passed to a 1L volumetric flask. The solution was kept under magnetic stirring for 1 h, to ensure that the salts have completely dissolved.

After one hour of stirring, the magnetic rod was removed from the volumetric flask and the flask is levelled to obtain 1L of buffer. Finally, the buffer was filtered through a sterile filter of 0.22 μm (Top-Filter Nalgene, 90 mm, pore 0.2, 500 mL, thread GL45) with the help of a vacuum pump.

The preparation of the hydrogel was carried out inside the laminar flow cabinet located in a cleanroom. Table 2 below details the composition of the P407 hydrogel in units of g/L.

TABLE 2
Composition of the P407 hydrogel in units of g/L

| P407 Hydrogel | Gel P407—14.73% (w/w) | |
|---|---|---|
| Composition | (g/L) | Function |
| POLOXAMER ® 407 | 173 | Thermogelling |
| | | agent |
| Hydrogen phosphate | 0.6 | Buffer pH |
| di-sodium dodecahydrate | | |
| Dihydrogen sodium | 0.05 | Buffer pH |
| phosphate dehydrate | | |
| Sodium chloride | 0.4 | Osmolarity |
| | | modifier |
| Water for Injection (WFI) | 1000 (no QS to | Solvent |
| | 1000 mL) | |

P407 14.73% (w/w gel) was prepared by the slow addition of P407 to a cold buffer solution (NaH₂PO₄.2H₂O 0.05 g/L, NaHPO₄.12H₂O 0.6 g/L, NaCL 0.4 g/L, pH 7.4), and maintained on a roller stirrer at 4-8° C. for 6 h.

To prepare 100 mL of the hydrogel, first a sterile 250 ml borosilicate glass lab bottle was placed in the precision balance and it was tared. Then, 17.3 g of P407 were weighed in the tared bottle and 100 mL of the previously prepared cold phosphate buffer was added. Finally, the solution was stirred, initial strong stirring was carried out for 60 seconds to facilitate the dissolution of P407, and then it was kept under stirring on a roller stirrer at 30 rpm for 6 hours in a refrigerator. After 6 h, the P407 was completely dissolved, and was left in the refrigerator overnight so that the foam generated during the stirring process will disappear.

The hydrogel was stored in a refrigerator at a temperature between 2° C. and 8° C., until use.

The loading of LPT99 was produced by forming a homogeneous solution of the drug in the P407 14.73% w/w vehicle. Briefly, to prepare 20 ml of a 300 μg/mL solution of LPT99 in P407 14.73% (w/w) gel, a sterile 20 ml amber glass vial was first placed on the precision balance and it was tared. Then, 6 mg of LPT99 was weighed in the tared vial and 20 ml of the previously prepared cold P407 14.73% w/w vehicle was added. Finally, in order to obtain a solution as homogeneous as possible, it was stirred in an ultrasonic bath for a time frame between 40 seconds and 60 seconds, until a homogeneous and free of lumps solution was obtained.

Samples were kept under refrigeration (typically 4° C.) and resuspended before using.

Once the solution of LPT99 was prepared and homogenized, it was dosed in different vials for later use. It was very important to keep vials cold during dosification. If the product thickens during preparation, the vial should be placed back in the refrigerator. The vial should be held by the cap to prevent gelation due to temperature transition.

Before dosing the product from one vial to another, e.g. empty sterile vial, the vial containing the solution was shaken to mix its contents until a visually homogeneous solution was obtained. Then, with the aid of a micropipette, the solution was pipetted several times to mix and withdraw a homogeneous sample, and 1000 μL of the solution was removed and added to the empty sterile vial. This action was repeated each time the sample was withdrawn. It is important to hold the vial by the cap to prevent gelation.

### Example 2: Analysis of the Final Product

The viscosity measurement of the hydrogel was performed to determine the behavior of the viscoelastic agent once gelled within the ear. The measurement was carried out following the European Pharmacopoeia Method, section 2.2.10 (measured at 37° C., body temperature).

P407 is a thermoreversible compound, existing in a liquid or gel state depending on its temperature. Accordingly, it can form a semi-solid gel at body temperature of 37° C., being liquid at room temperature. During the development process, this allowed formation of an easy to handle solution with the LPT99 molecule, which gels at 37° C., once the solution is administered to a patient.

Viscosity measurements were performed at 37° C. to simulate the real conditions of application of the gel, once it has gelled. The solution was first placed in a climatic chamber at 37° C. to gel (20 mL of solution for about 1 h), and once gelled, the viscosity was measured by maintaining this temperature with a thermostat bath (temperature control equipment for viscosity measurements). The gelation of the product was carried out in a 20-mL syringe to facilitate the incorporation of the gel into the sample chamber of the viscometer, once gelled.

The viscosity measurement was performed with a Rotational Viscometer (FungiLab/Evo Expert). The viscosity measures vary depending on the temperature. That is why the temperature was controlled by the temperature probe of the viscometer, keeping it at 37° C. To achieve this temperature, the viscometer was connected to a thermostat bath. The equipment used depends on the viscosity of the gel. This viscosity determines the spindle and adapter to be used. Also depending on the spindle used, a quantity of sample is required as well as a speed of rotation (RPM) to reach SR=1s−1.

The following equipment was used:

| | | | | sample | |
|---|---|---|---|---|---|
| P407 | | | | volume | RPM |
| concentration | Viscosimeter | Adapter | Spindle | (mL) | (SR≈1s-1) |
| 14.73% (w/w) | EvoExpert R | APM | TR11 | 13.5 | 4 |
| | (10026) | (small | | | |
| | | sample | | | |
| | | adapter) | | | |
| | | with | | | |
| | | thermo- | | | |
| | | statation | | | |
| | | jacket | | | |

The sample chamber of the low sample amount adapter was filled with 13.5 ml sample. After filling the sample chamber, the spindle was inserted (TR11 in this case). Since the penetration of the spindle alters the surface of the gel, it was necessary to allow the sample to stabilize before measuring (approximately 30 min). The sample should be free of bubbles, as these could distort the measurement. The measurements were carried out at 1 s-1 shear rate (SR). For this, the spindle is programmed so that it turns to the corresponding RPM (4 rpm in this case). Finally, the measurement time was programmed (in seconds, 3600 sec equivalent to 1 h of measurement) and after that time a graph showing the viscosity (cp) versus time (sec) at 1 s⁻¹ SR at 37° C. is obtained.

pH measurements were performed to ensure that it is maintained in the physiological range for the indicated application. A Crison pH-meter was used and the measurement was carried out following the European Pharmacopoeia Method, section 2.2.3, after calibrating the equipment following the indications of the apparatus. The pH should be maintained in the range 7-7.5, most preferably 7.2.

Osmolality measures were performed to ensure that it is maintained in the physiological range for the indicated application. The determination of the Osmolality was carried out by means of a cryogenic osmometer following the European Pharmacopoeia Method, section 2.2.35, and is preferably maintained in the range between 240 mOsmol/kg and 350 mOsmol/kg.

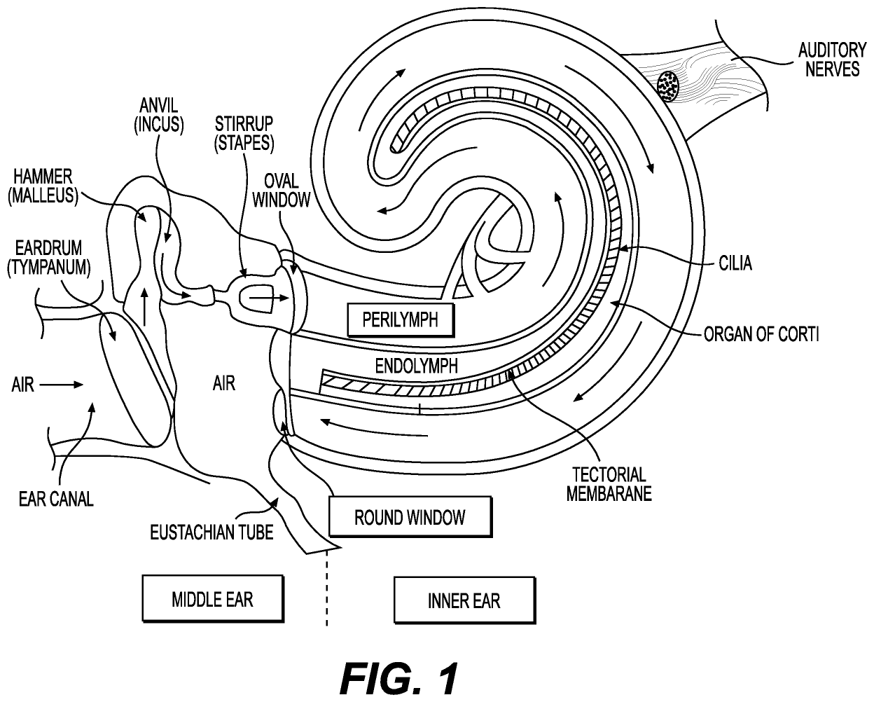

The in vitro release assay was performed using cellulose dialysis membranes of 3500 Da (OrDial D35-MWCO 3500, Orange Scientific) to simulate the round window membrane, located between the middle ear and the inner ear, as it is the first barrier for the drug to reach the inner ear where it will exert its pharmacological action. (See FIG. 1) Artificial perilymph (NaCl 137 mM, KCl 5 mM, CaCl₂ 2 mM, MgCl₂ 1 mM and NaHCO₃ 1 mM) was used to simulate the environment inside the inner ear, since this is the liquid that interact swith the drug after crossing the round window. The assay was performed at 37° C., continuing with the simulation of ear conditions.

Table 3 details the composition of the artificial perilymph in units of g/L:

TABLE 3
Composition of the artificial perilymph in g/L

| Composition | (g/L) |
|---|---|
| Sodium chloride | 8.006 |
| Potassium chloride | 0.373 |
| Calcium chloride | 0.222 |
| Magnesium chloride | 0.095 |
| Sodium bicarbonate | 0.084 |
| Type II Water | c.s.p. 1 L |

For the preparation of the artificial perilymph, the reagents were weighed one by one on a precision balance. A 250 ml beaker was placed with around 150 ml of Type II Water and kept under magnetic stirring. The reagents were then added as follows: first, 8.006 g of sodium chloride was weighed into an aluminum weighing pan (WPAL-072-100) and added to the Type II water being stirred. To ensure that everything was added, the rest of the reagent that remained on the weighing pan was washed with water. The same procedure was followed to add the rest of the reagents. After all reagents were weighed and added, the buffer was kept under magnetic stirring in the beaker for 15 minutes. Next, the solution was passed to a 1L volumetric flask and type II water was added but without levelling the flask. The solution was kept under magnetic stirring for 1 h, to ensure that the salts were completely dissolved. After one hour of stirring, the magnetic rod was removed from the volumetric flask and the flask was leveled to obtain 1L of artificial perilymph.

To prepare the release assay, ten (10) mL of the LPT99 loaded hydrogel was placed inside a 3500 Da dialysis membrane, the membrane with the hydrogel was introduced into a 100 ml borosilicate glass lab bottle and 30 ml of artificial perilymph was added. All samples were kept under magnetic stirring at 37° C. Due to the thermo-reversible behavior of P407, firstly the hydrogel was deposited into the membrane at 37° C. and left to harden. Once it gelled, the artificial perilymph was added and kept under stirring at 37° C.

Samples were taken at different time points (1 h, 3 h, 6 h, 1 day, 2 days, 3 days, 6 days, 7 days, 8 days, 9 days, 10 days, 13 days, 14 days and 15 days) at which time 5 mL were withdrawn with a graduated glass pipette and replaced with equivalent volume of artificial perilymph. The collected samples were analyzed to determine the amount of drug released from the hydrogel at each time point.

Analytical Method for the Quantification of the Released LPT99

For the analysis of LPT99 released from the hydrogel, samples analysis was performed using a High-Performance Liquid Chromatography with Diode-Array Detection (HPLC/DAD) (Agilent) with a calibration curve in the range of 0.2-20 ppm. The collected samples were previously purified using solid phase extraction C18 (SPE C18) cartridges.

LPT99 molecule presents spectroscopic activity in the UV range, at wavelengths between 200 nm and 280 nm. Quantification by HPLC-DAD is a suitable method in the absence of interfering compounds.

For the LPT99 analysis, an Agilent 1290 Infinity UHPLC liquid chromatograph (Agilent Technologies, Waldbronn, Germany) equipped with a diode array detector (DAD), an autosampler, an automatic injector, and a column oven were utilized. As stationary phase, a Zorbax Eclipse Plus C18 rapid resolution column (50×2.1 mm, 1.8 μm particle size, Agilent) guarded with an in-line filter (0.3 μm pore size frit, 2.1 mm diameter, Agilent) kept in a column oven at 30° C. was used. Water (A) and acetonitrile (B), each containing 0.1% formic acid (v/v), served as mobile phases eluting at a flow rate of 0.6 ml/min. The gradient was t=0.0 min, /0% A; t=0.3 min, 70% A; t=7 min, 30% A; t=8.5 min, 30% A; t=9 min, 70% A; t=10 min, 70% A. Between runs, the column was equilibrated with 70% A for 1 min. The injection volume was 1μl and chromatograms were recorded at 230 nm and 278 nm.

Calibration Curve Prepared Directly in MeOH

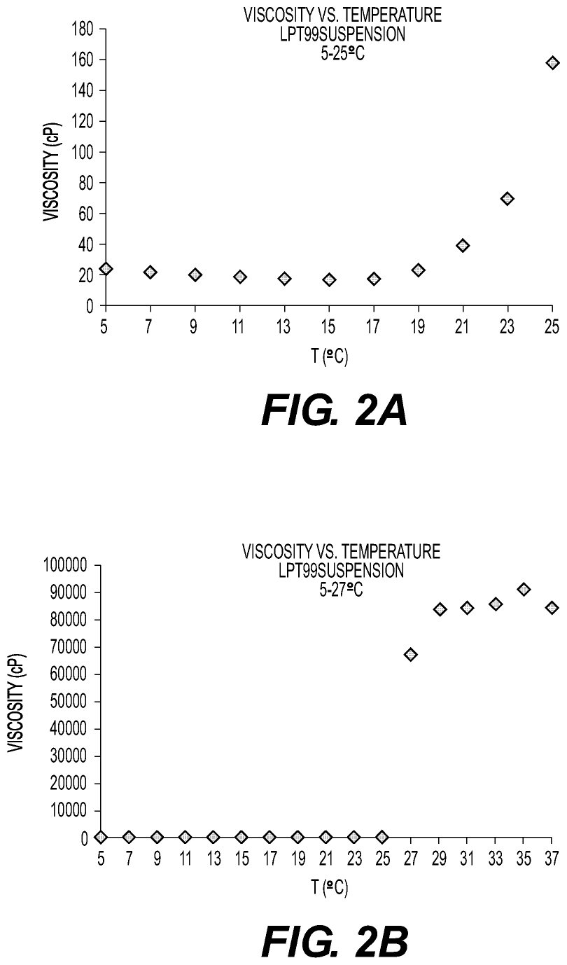

For direct LPT99 quantification, an external calibration curve of LPT99 was prepared in methanol (MeOH) in the range of 0.2-25 ppm, starting from a 100 ppm stock solution which was diluted with MeOH to obtain various standards of the curve. The analysis of the blank showed no absorbance in the range of the considered wavelengths. The coefficient of regression of the curve was R2=0.996 indicating good linearity in the concentration range tested. (See FIG. 2B) This quantification method was therefore appropriate if the sample is free of interference, so a suitable extraction process was necessary.

Solid Phase Extraction (SPE)

This method was performed for extracting LPT99 from perilymph samples (samples of perilymph with the LPT99 released from the hydrogel during the in vitro release assays).

The separation was performed using Hypersep C18 solid phase extraction cartridges (500 mg, 3 mL) from Thermo scientific (Rockwood, USA). Conditioning of the cartridge was carried out with methanol (MeOH), followed by cleaning the samples by H2O. The drug was eluted with MeOH, evaporated and reconstituted with MeOH for its quantitation by HPLC-DAD.

The extraction method was optimized by adjusting the load volumes to ensure that the amount of drug retained in the stationary phase was the highest possible. The volumes of water used in the cleaning phase were adjusted to ensure an effective elimination of interfering components avoiding the loss of retained analyte. Finally, the volume of MeOH used as eluent was adjusted to achieve a complete elution of LPT99 in the smallest possible volume, thereby causing the pre-concentration of the analyte and an improvement of the signal obtained in the HPLC-DAD.

A vacuum manifold from Varian (Palo Alto, USA), connected to a vacuum pump was used for the solid phase extraction (SPE) process. Before analysis, dry cartridges were first conditioned by percolating 5 mL of methanol, followed by 5 mL of water. Five (5) mL of sample (or standard) were subsequently loaded and cartridges were then washed with 20 mL of water, in order to remove the remaining polymer. The target compound was recovered eluting the SPE column with 1 mL of methanol.

Calibration Curve Prepared after the Extraction Process

A calibration curve was prepared in the concentration range of 0.2-20 ppm starting from a 500 ppm stock solution, but it was diluted with perylimph from a pool of blank samples (absence of LPT99) to obtain various standards of the curve. These standards were subjected to an extraction process using HYPERSEP® C18 solid phase extraction cartridges (500 mg, 3 mL) from Thermo scientific (Rockwood, USA). To avoid any solubility problem of the target compound in perylimph, 0.5 ml methanol was added to the standards and the samples before extraction. The coefficient of regression of the curve was R2=0.999 indicating good linearity in the concentration range tested.

Results

FIGS. 2A and 2B demonstrate the formation of thermoset gels under in vivo conditions, showing how the formulation goes from a liquid which can be administered by injection at room temperature (15-25° C.) to a semi-solid hydrogel at body temperature (37° C.).

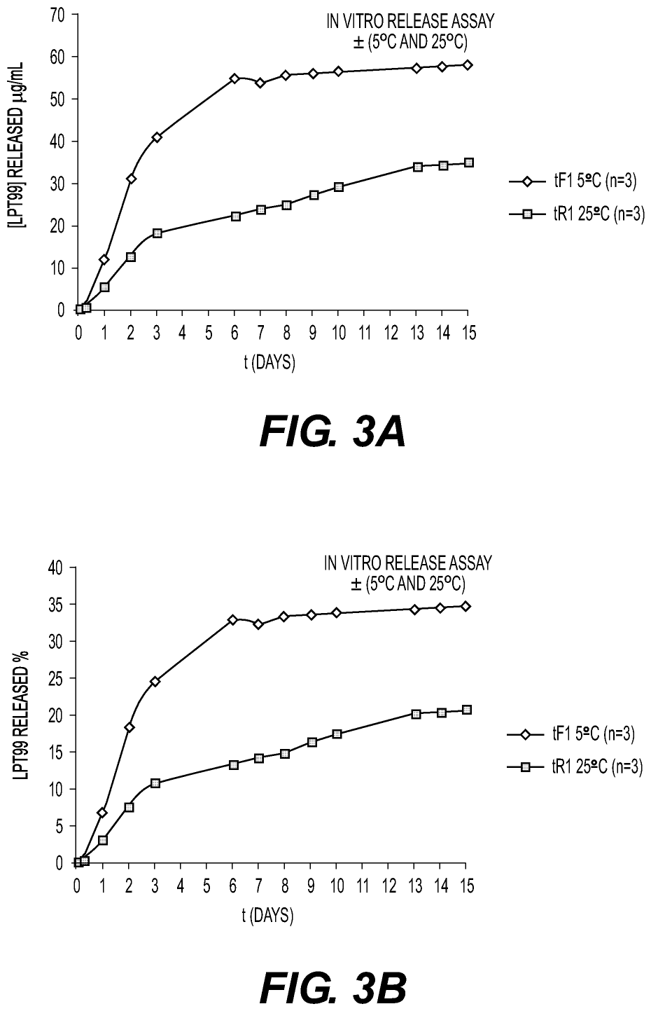

FIGS. 3A and 3B demonstrate that the drug, LPT99, is released over time (days) in a controlled manner. FIG. 3A shows release as a function of amount (μg/ml). FIG. 3B shows release as a percent of total drug. The drug is released in effective amounts for at least one week.

### Example 3: In Vitro Studies Showing Efficacy of Formulation

Materials and Methods

The specificity of LPT99 was tested in vitro on Apaf1, caspase 3, and caspase 9 (proteins from the apoptotic cascade), and a broad panel of potential pharmacological targets.

To identify off-target activities of LPT99, its selectivity against a panel of receptors (44 G-protein-coupled receptors [GPCR] and 4 non-GPCR), 4 ion channels, and 3 transporters were analyzed. The cell line, HEI-OC1 (house ear institute organ of Corti 1), which expresses several characteristic markers of the organ of Corti sensory cells (Kalinec, et al. , Audiol. Neurootol. 2003, 8(4), 177-89), was used to evaluate the efficacy of LPT99 in preventing apoptosis due to CisPt prevention. Cells were pre-incubated with LPT99, followed by CisPt prevention for 24 hours. Under these conditions, the 50% inhibitory concentration (IC50) for caspase 3 was 5.2±1.6 μM. Both LPT99 stereoisomers were equally effective and equivalent to the racemic mixture, suppressing caspase 3 activation in the cellular model.

Flow cytometry was also used to characterize the effect of LPT99 in the release of mitochondrial cyt c in HEI-OC1 cells in vitro with CisPt prevention. Results

LPT99 inhibited the formation of the apoptosome complex composed by recombinant Apaf1, cyt c, deoxyadenosine triphosphate (dATP), and caspase 9. This activity was measured as inhibition of caspase 3 activation. At 10 μM, the LPT99 apoptosome inhibition was (mean % ±standard deviation [SD] %) 78.9% ±12.7%. To evaluate the specificity of the inhibition, an assay of caspase 3 and 9 activation was set up with recombinant proteins. The inhibitions of caspase 3 and 9 were 7.6±14.7 and 5.3±3.5, respectively, for LPT99. These results probed the specificity of LPT99 on Apaf1 inhibition among other components of the apoptotic cascade.

The in vitro inhibition obtained for 41 of these tested targets was <50% at 10 μM, indicating that LPT99 had no affinity for them. For 14 of the tested targets—adenosine receptor 3 (A3); cholecystokinin: cholecystokinin receptor 1 (CCK1); melatonin receptor (MT1); neurokinin (NK2 and NK3); opioid (kOP and mOP); serotonin receptors (5HT-1A, 5HT-2A, 5HT_{-2B} and 5HT₋₇); and vasopressin (V1A; Na+channel site 2 and Cl-GABA-gated channel)—the LPT99 affinity was >50%. At 1 μM, the inhibition of 13 of these targets was <50%, indicating a very low affinity with LPT99. Inhibition of MT1 was 53% at 1 μM and 6% at 0.1 μM, showing a low affinity of LPT99 for this protein. These results confirmed the LPT99 specificity for Apaf1 inhibition. Moreover, because no LPT99 has been found in plasma after TT administration of the LPT99 formulation in a hydrogel; detection limit at 2 ng/mL=3,2 nM), no side effects due to these low-affinity interactions were expected.

In vitro experiments with a 2,5-piperazinedione derivative showed suppression of caspase 3 activation in vitro, distribution to the cochlea after intratympanic administration in a dose dependent manner, and protection from apoptosis as well as maintenance of cell viability after CisPt prevention. In vitro experiments with LPT99 demonstrated the drug's specificity for Apaf1, resulting in its inhibition.

In a cellular model of CisPt-induced apoptosis, LPT99-treated cells showed a decreased release of cyt c from mitochondria, reduced caspase-3 activation, and improved cell viability, showing the cytoprotective effect of LPT99.

LPT99 inhibited release of mitochondrial cyt c from 28% ±6.6% after CisPt prevention to 68.1% ±1.0% in cells that had been pre-incubated with LPT99). This dual inhibitory effect of LPT99 resulted in increased cellular viability with CisPt prevention. Prevention with CisPt (0 to 5 μg/mL) resulted in a dose-dependent decrease in HEI-OC1 cell viability (IC₅₀=4.47±1.94 μg/mL). Survival rate increased in the presence of 1 μM LPT99, with an IC₅₀ of 10.51±3 μg/Ml. The effect of LPT99 on proliferation of non-apoptotic cells was studied. A549 cells were cultured in the presence of LPT99 for up to 6 days; the cell number was monitored by flow cytometry, and doubling time was calculated. The Apaf1 inhibitor delayed cellular proliferation by accumulation of cells at the G1 phase of the cell cycle; if the Apaf1 inhibitor was removed from the medium, this effect was reversible. These results indicated that Apaf1 pharmacological inhibition in nonapoptotic cells did not increase the cellular proliferative rate in vitro.

### Example 4: Dose Development

Materials and Methods

During nonclinical development studies, the following dose nomenclature for LPT99 was presented, as shown in Table 4.

TABLE 4
Dose nomenclature for LPT99 solution

| Dose (μg/mL) | Equivalent Dose (μM) |
|---|---|
| 32 | 50 |
| 63 | 100 |
| 300 | 478 |
| 500 | 797 |

Results

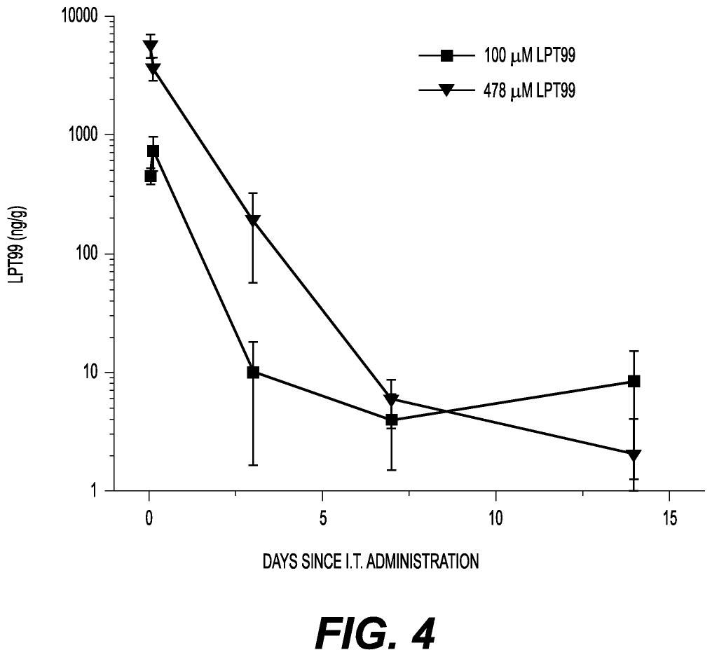

In vivo experiments in rats showed that, after trans-tympanic (TT) administration of LPT99 in hydrogel, LPT99 distributed locally to the cochlea. See FIG. 4. The safety of TT administration was also confirmed, as LPT99 levels that could offset CisPt efficacy were not detected systemically. LPT99 TT administration protected against CisPt-induced hearing loss, when compared with the vehicle control This effect was dose dependent; the group prevented with CisPt plus LPT99 showed significantly lower auditory threshold shifts than seen in the CisPt control group.

### Example 5: In Vivo Efficacy in Preventing Hearing Loss

Materials and Methods

A model of CisPt-induced hearing loss in rats was evaluated to test the in vivo efficacy of LPT99. Ototoxicity was induced by intraperitoneal (IP) slow infusion of CisPt at doses that compared with those used in human preventions (eg, 10 mg/kg). LPT99 was administered TT 30 minutes before CisPt was given. LPT99 was prepared in 2 compositions: a solution in 5% HPβCD in physiological serum (LPT99-CD), and a POLOXAMER® 407-based thermoreversible hydrogel (LPT99 solution).

The protective effect of LPT99 was evaluated 3 days after CisPt administration by functional measures, such as auditory brainstem response (ABR) threshold shift, DPOAE, and expression of biomarkers of apoptosis in cochlea and cytocochleograms.

Results

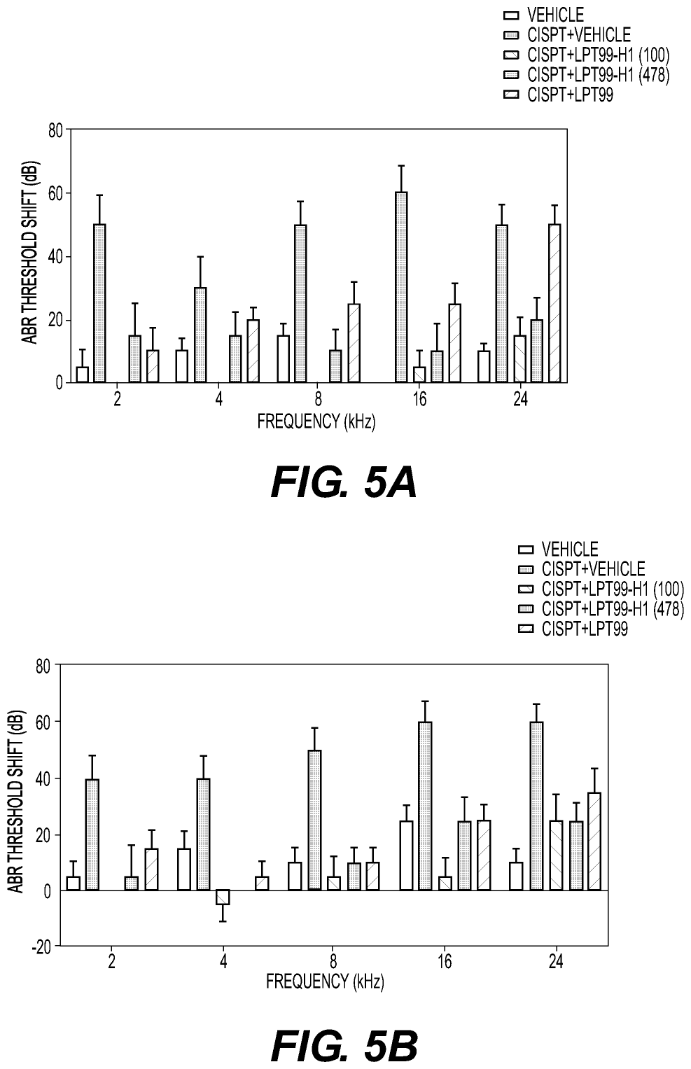

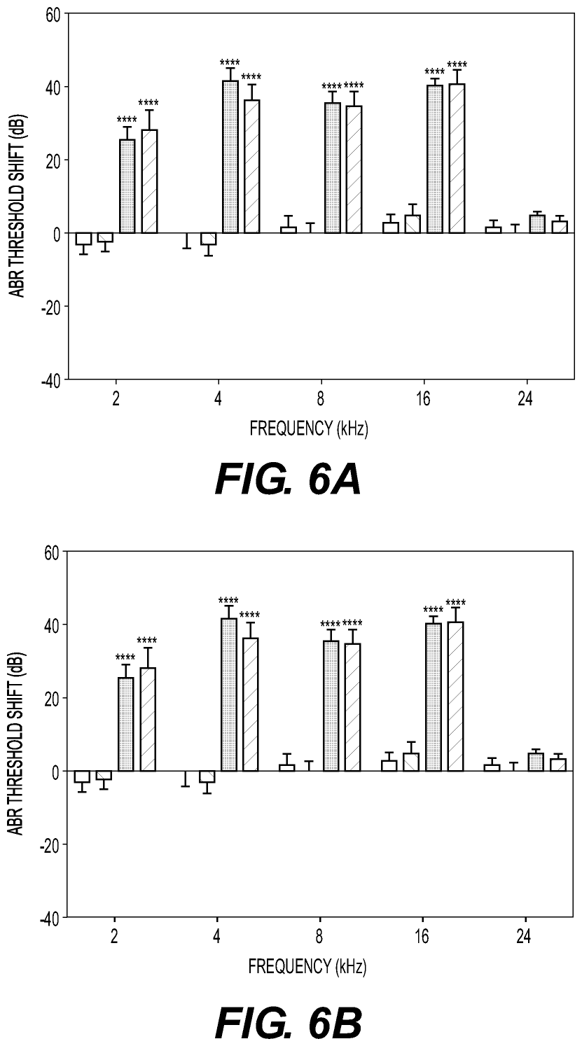

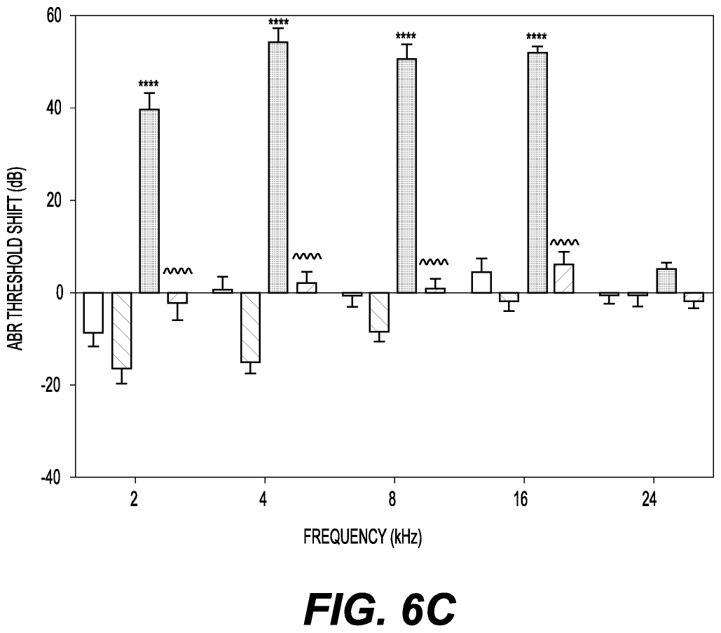

Transtympanic administration of LPT99-CD at 3 doses (50, 100 and 200 μM) was protective against CisPt-induced hearing loss, as determined by ABR. See FIGS. 5A and 5B. Administration of LPT99 attenuated the ABR threshold shift induced by CisPt at 3 days after administration, especially for high frequencies (20, 28, or 40 kHz).

This protective effect was dose dependent, showing that the 100 μM dose had the best protection profile. In addition, LPT99-CD diminished the changes induced by CisPt administration in ABR amplitudes (indicating the number of firing neurons) and peak latencies (indicating transmission speed). LPT99-CD significantly reduced the expression of p53, compared with the non-prevented cochlea.

Administration of CisPt-induced kidney injury molecule-1 (Kim-1) expression in the rat cochlea (Mukherjea, et al., Neuroscience 2006, 139(2), 8), with Kim-1 considered a marker of ototoxicity. In the tested model, it was found that Kim-1 expression decreased with LPT99-CD prevention.

Transtympanic administration of LPT99 solution at 2 doses (63 μg/mL [100 μM] and 300 ug/mL [478 μM]) was also protective against CisPt-induced hearing loss. At 3 days after CisPt administration, ABR thresholds had significantly increased, and DPOAE amplitudes had significantly decreased. A massive outer hair cell (OHC) loss was seen in the medial-basal parts of the cochlea, as determined by cytocochleogram analysis. The CisPt-induced increase of ABR threshold, decrease of DPOAE amplitudes, 7 and OHC cell loss were significantly prevented by TT administration of LPT99 solution, at both (63 and 300 μg/mL) doses. These results shown in FIG. 4 demonstrated significant protective effects of LPT99 solution on auditory function.

### Example 6: Safety Pharmacology

Materials and Methods

In a study conducted in female Wistar rats, plasma concentrations of LPT99 were evaluated at 1 hour and 2, 4, 7, and 15 days after TT administration of a single 50 μL dose of 50, 100, or 200 μM LPT99 formulated in a 5% HPβ-cyclodextrin (“CD”) vehicle (LPT99-CD).

A functional observation battery was conducted with LPT99 administered via IP (intraperitoneal) injection in Sprague-Dawley rats. The study was preceded by a non-GLP maximum tolerated dose (MTD) toxicity study, via the same administration and test system, which determined the MTD of LTP99 to be 1000 mg/kg. LPT99 was suspended in vehicle [0.5% w/v methylcellulose and 0.1% v/v TWEEN®80 in Milli-Q water] and administered intraperitoneally to Sprague-Dawley rats as a single dose at the doses of 100 (low—G2/G2TK), 300 (mid—G3/G3TK), and 750 (high—G4/G4TK) mg/kg body weight. The rats in vehicle control groups (G1/G1TK) received the vehicle alone. The dose volume administered was at an equivolume of 10 mL/kg body weight for all groups.

The potential cardiotoxicity of LPT99 was investigated in an assay using the hERG-CHO cells transfected with the automated patch clamp assay.

Results

Plasma LPT99 concentrations were below the lower limit of quantitation (BLLQ, 2 ng/mL 3.2 nM]) at all evaluated time points. A plasma concentration of 3.2 nM LPT99 corresponded to 0.006%, 0.003%, and 0.0016%, respectively, of the administered 50, 100, and 200 μM doses.

These bioavailability values are similar to those described for other drugs formulated in POLOXAMER® gels and administered TT (Honeder, et al., Audiol. Neurootol. 2014, 19(3), 193-202; Wang, et al., Audiol. Neurootol. 2011, 16(4), 233-41; Yang, et al., Sci. Transl. Med. 2016, 8(356), 356ra120). Assuming a maximum clinical dose of 200 μg LPT99 and an average human plasma volume of 2400 mL (for an average 60-kg individual [typical blood plasma volume in males is ˜39 mL/kg of body weight, and in females ˜40 mL/kg]). If 100% of the TT dose were 100% bioavailable, the plasma LPT99 concentration would be 0.066 μM, below the IC₅₀ (5.2±1.6 μM.

The neurological parameters were unaffected by the prevention at 100 mg/kg dose on Day 1. At 300 and 750 mg/kg/day, test item-related lower motor activity scores were observed in both sexes on day 1 and reversible by Day 15 and hence considered non-adverse effects.

The IC₅₀ of LPT99 was 3.4×10⁻⁶M. LTP99 was not detectable in plasma after TT administration, and the LPT99 detection limit of the analytical method was 3.2 nM; thus, the safety margin was >1063.

An in vitro experiment evaluating the effects of LPT99 on ion channels, including hERG potassium channels, in CHO and HEK 293 cells, LPT99 had a low torsadogenic risk. Torsadogenic refers to the development of torsade de Pointes (TdP) arrhythmias

### Example 7: Pharmacokinetics and Product Metabolism in Animals

The routes for drug entry into the inner ear include the systemic circulation and the round window membrane (RWM), which connects the middle and inner ears (El Kechai, et al., Int. J. Pharm. 2015, 494(1), 19). In the case of an apoptosis inhibitor, avoiding the systemic route is of crucial importance, as this prevents any interaction with CisPt antineoplastic activity outside the cochlea. Transtympanic LPT99 administration is an efficient and less toxic alternative route to systemic delivery.

As described above and shown in FIG. 4, after TT administration to rats, LPT99 distributed to cochlea in a dose-dependent manner No distribution to the contralateral cochleae or plasma was observed, which suggests that LPT99 distributes locally to the administered cochlea.

Cochleae were harvested and rinsed at several timepoints after intratympanic injection of drug product. Drug concentration (y axis) in cochlear homogenates is expressed as nanograms LPT99 per gram of cochlear homogenate.

(i) Absorption

LPT99 cochlear distribution after TT administration was studied in rats. LPT99-CD (50, 100, or 200 μM) or LPT99 solution (100 and 478 μM) was administered, and plasma samples and cochleae were collected at 1, 3, and 24 hours (for LPT99-CD), or at 1 and 3 hours and 1, 3, 7, and 14 days (for LPT99 solution) post-prevention. LPT99 concentration was quantified with an ultra-performance liquid chromatography tandem mass spectrometry (UPLC-ESI/MS/MS) system.

LPT99 was detected in all cochleae at 1 and 3 hours (LPT99-CD), and at 1 and 3 hours and 1, 3, 7, and 14 days (LPT99 solution) post-prevention.

At the 50, 100, and 200 μM doses, peak mean cochlear LPT99 concentrations of 328.5, 491.3, and 611.1 ng/g cochlea, respectively, were seen at 1 hour post-prevention. At 24 hours post-prevention, the mean cochlear LPT99 concentrations had decreased to 1.8, 13.9, and 13.4 ng/g cochlea, respectively.

In contrast, LPT99 concentrations in the contralateral cochleae and plasma were BLLQ (3 ng/g and 2 ng/mL, respectively) at all time-points and doses. These results indicated that LPT99 distributed locally to the administered cochlea and confirmed the safety of TT administration, because LPT99 levels that could offset CisPt efficacy were not detected in plasma.

(ii) Distribution:

Distribution of LPT99 was investigated in cochlea and plasma after a single TT administration in rats. The inhibitor was administered at 50, 100, or 200 μM (dissolved in 5% hydroxypropyl cyclodextrin in physiological serum).

LPT99 was detected in all administered cochleae at 0.5 and 1 hour post-prevention, showing a direct correlation between product dose and concentrations in the cochleae. Contralateral cochleae and plasma presented concentrations of LPT99 that were BLLQ at all time points.

(iii) Metabolism

The metabolic profile and stability of LPT99 was characterized in human, dog, rabbit, rat, and mouse microsomal and S9 fractions, and in human cytosolic fractions.

LPT99 was extensively metabolized (>90% metabolized after 1 hour) in microsomal and S9 hepatic fractions in all tested species. Up to nine (9) metabolites were formed through phase I biotransformation pathways. The most abundant metabolites were identified as single hydroxylation, double hydroxylation, and demethylation plus double hydroxylation. The quantities of metabolites formed by both compounds in dog, rat, and mouse species showed similar patterns to that seen in humans In the rabbit S9 fraction, the quantity of detected metabolites was less than in the human S9 fraction.

(iv) Phototoxicity

An in vitro experiment was performed in BALB/c 3T3 mouse fibroblasts to determine the phototoxic potential of LPT99 at concentrations of ≤100 μM in 3T3L1 cells.

LPT99 was not cytotoxic in the presence or absence of ultraviolet-visible irradiation, as indicated by the absence of a calculable photo-irritation factor. Thus, LPT99 was found to be not phototoxic.

### Example 8: Single-Dose Toxicity Study—Auditory Toxicity of LPT99 after Transtympanic Administration in the Rat

The potential ototoxicity of LPT99 solution was investigated in two Non-GLP studies.

An initial experiment with LPT99 solution at concentrations of 100 μM and 200 μM showed no LPT99 ototoxicity. A follow-up experiment with LPT99 at 100 and 478 μM similarly showed no LPT99 ototoxicity.

LPT99 at 200, 400, and 797 μM showed no ototoxicity.

### Example 9: Single-Dose Toxicity Study of LPT99 after Intraperitoneal Administration in the Rat Acute Systemic Toxicity Model

A GLP acute systemic toxicity study was conducted with LPT99 administered via IP injection in Sprague-Dawley rats. The acute systemic toxicity study was preceded by a non-GLP maximum tolerated dose (MTD) toxicity study, via the same administration and test system, which determined the MTD of LTP99 to be 1000 mg/kg.

Materials and Methods

LPT99 was suspended in vehicle [0.5% w/v methylcellulose and 0.1% v/v TWEEN® 80 in Milli-Q water] and administered intraperitoneally to Sprague-Dawley rats as a single dose at the doses of 100 (low—G2/G2TK), 300 (mid—G3/G3TK), and 750 (high—G4/G4TK) mg/kg body weight. The rats in vehicle control groups (G1/G1TK) received the vehicle alone. The dose volume administered was at an equivolume of 10 mL/kg body weight for all groups.

The main toxicity groups consist of 15 rats/sex/group in G1 and G4 groups and 10 rats/sex/group in G2 and G3 groups. The toxicokinetic groups consisted of 6 males and 6 female rats each for the prevention groups, whereas the vehicle control group had 3 male and 3 female rats.

Results

Findings from this study showed that LPT99 administration via IP injection was generally safe and well tolerated. There were no clinical signs observed in all the tested dose groups. No mortality was observed.

Toxicokinetic assessment indicated that the time to reach peak plasma concentrations (Tₘₐₓ) of LPT99 was observed at 24 h (except 2 h and 8 h in male and female at 100 mg/kg/day dose level) and plasma concentrations were quantifiable till 24 h at all the tested dose levels in both genders. More than dose proportional increase in peak plasma concentration (Cₘₐₓ) and exposure (AUCₗₐₛₜ) was observed from 100 mg/kg/day to 750 mg/kg/day in male and approximate dose proportional increase observed in female from 100 to 750 mg/kg/day dose levels. Gender related differences were observed. Females showed approximately 1.5-5.5 fold higher exposure at all tested dose levels.

The neurological parameters were unaffected by the prevention at 100 mg/kg dose on Day 1. At 300 and 750 mg/kg/day, test item-related lower motor activity scores were observed in both sexes on day 1 and reversible by Day 15 and hence considered non-adverse effects.

At 750 mg/kg, body weights or body weight gains were not statistically significantly lower during first 7 days after injection in both sex. However, there was tendency to gains in body weights from Day 4 till the end of life, indicating reversal of the test item-related effects. At 750 mg/kg, statistically significant reduction in the food consumption was observed in males (during days 1-7) and females (during days 1-4) when compared to the control group.

LPT99 induced changes on Day 2 indicated increased neutrophil count in all prevented groups and at 750 mg/kg, this increase was also associated with increased lymphocyte and leukocyte counts in males. This increase was attributed to the acute inflammatory response around the injected material and this change did not show any microscopic correlates in hemopoietic organs.

LPT99 induced changes on Day 15 included a minimal increase in neutrophil count noted in 750 mg/kg dose group males. This increase in cell count did not show microscopic correlates in hemopoietic organs. The coagulation parameters were not affected by test item administration on both Days 2 and 15. There were no test item related changes in clinical chemistry parameters in males. In females, an increase in triglyceride concentration (60% to 147%) was noted in all prevented groups on Day 2. This change was considered as a test item related transient finding as this finding was not present on Day 15. The urinalysis parameters were unaffected by test item administration on both Days 2 and 15.

The terminal fasting body weights were not affected by test item administration at both the intervals (Day 2 and 15). On Day 2, an increase in liver weight was noted in males and females at 300 and 750 mg/kg dose groups. This increased weight was associated with the microscopic finding of hepatocellular hypertrophy and considered as an adaptive metabolic change to test item administration. An increase in epididymides weight was present at 750 mg/kg dose group males. This weight increase was attributed to the test material deposit in the epididymal fat as well as on the capsule.

Results on Day 2 indicated test material was deposited in the abdominal cavity (mesentery) which was observed over the surface of different abdominal/pelvic cavity organs namely liver, pancreas, kidneys, adipose tissue, epididymal fat and capsule, testes, seminal vesicles and coagulating gland, different intestinal segments and abdominal muscle. The grossly observed white foci/material were microscopically confirmed as the eosinophilic material surrounded by cell debris and inflammatory cells consisting mainly of neutrophils. Mediastinal lymph node white discoloration was present in all test item injected groups and microscopically necrosis/inflammation of lymph node was noted with presence of eosinophilic injected material. This could be consequent to the peritoneal space lymphatic drainage via thoracic duct to mediastinal lymph nodes.

On Day 15, as observed on Day 2, white discoloration/foci were noted in all the abdominal organs, diaphragm and mediastinal lymph nodes. However, the distribution was limited when compared to the gross lesions noted on Day 2. The microscopic morphology of these white foci also differed from Day 2. The volume of eosinophilic material was less and was surrounded predominantly by macrophages and mononuclear cells with decreased neutrophil population indicating a chronic inflammatory response.

In mediastinal lymph nodes, increased number of foamy macrophages were present without displacement of lymphoid tissue with the injected material.

At both the intervals (Day 2 and Day 15), the inflammatory response was restricted to the mesentery and surface of the visceral organs in abdominal cavity and the parenchyma was not affected. On Day 15, the reduction/absence of cell debris and lower volume of injected material on the surface of visceral organs and absence of necrosis in mediastinal lymph nodes indicate a tendency for recovery in inflammatory process.

These results demonstrate LPT99 administered via IP injection in Sprague-Dawley rats was generally safe and well tolerated at the doses used in this study. There were no clinical signs observed in all tested dose groups and no mortality was observed. Drug related changes were generally attributed to the acute inflammatory response around the injected material and were reversible.

### Example 10: Genotoxicity

LPT99 showed no evidence of genetic toxicity in a GLP in vitro bacterial reverse mutation assay (Ames test).

Furthermore, in the in vitro Chinese hamster ovary (CHO) cell aberration assay, LPT99 did not induce structural aberrations in cultured mammalian cells, in the presence or absence of S9 metabolism.

The one major impurity in the active pharmaceutical ingredient, LPT102 was evaluated by quantitative structure-activity relationship ((Q)SAR) using the Derek Nexus and Leadscope Model Applier systems and identified as “inactive” for bacterial mutagenicity (SP21-17-FR).

### Example 11: Physical and Chemical Stability

The aqueous solubility of LPT99 at 2.4 micromolar (uM) and in 14.7 wt/wt % LPT99 is soluble at to at least 797 micromolar (a 332-fold increase in drug solubility). It was tested for physical and chemical stability.

### Physical Stability Studies:

Vials of the drug product were stored at either room temperature or 4° C. for a period greater than three months.

For vials stored at 4C, LPT99 precipitated after a period of approximately 2 months, as evidenced by cloudy solution or solid drug sediment. However, for vials stored at room temperature, solutions remained clear with no evidence of drug precipitation.

To test the LPT99 concentration in vials, the liquid drug product was filtered through sterile 0.2 micron filters.

For vials stored at 4C, less than 10% of the original drug concentration remained in solution, compared with 100% for vials stored at room temperature. Even physical methods of attempting drug reconstitution/dissolution (cycles of vortex, ultrasound) it was not possible to re-dissolve precipitated LPT99 in vials stored at 4C.

Viscosity of the drug product and the temperature induced phase change was unaffected by storage conditions tested up to and exceeding 3 months.

### Chemical Stability Studies:

Drug product stored at both 4C and room temperature demonstrates chemical stability up to and exceeding 3 months.

### Example 12: Local Tolerance

(i) Dermal Sensitization Study

A GLP-compliant dermal sensitization study was conducted with LPT99 in guinea pigs. The objective was to assess potential dermal sensitization of LPT99 using the Buehler test (Buehler, Arch Dermatol. 1965, 91, 171-7).

The test article, LPT99, was in a neutral-pH, buffered, isotonic solution containing a thermoreversible compound. The vehicle control was buffered at neutral pH and formulated as for the test article; it contained POLOXAMER® 407, disodium phosphate dodecahydrate hydrogen, sodium dihydrogen phosphate dihydrate, sodium chloride, and water for injection.

(ii) Range Finding Pilot Sub-Study

In the range-finding pilot substudy, 4 guinea pigs (2 males and 2 females; Charles River, Stone Ridge, N.Y.) were dosed. Guinea pigs were dosed topically in the left or right shaved scapula area with 50 μl of 1 of the 3 test article concentrations (200, 400, or 797 μM), or the vehicle control, to determine the highest nonirritating concentration that was well tolerated and that caused only mild-to-moderate irritation (i.e., a modified Draize Score of 1-2, described below) for the induction exposures. The modified Draize Score indicated separate assessments of the erythema and edema exhibited by each animal on a scale of Grade 0 to 4 for erythema and Grade 0 to 3 for edema.

No erythema or edema (both had Draize scores of 0) was observed for any of the tested LPT99 concentrations. Therefore, the highest LPT99 concentration 797 μM, was used for the induction and challenge exposure in the main study, because it was the highest nonirritating concentration used in the range-finding pilot substudy.

(iii) Dermal Sensitization Main Study

The main study consisted of 2 groups of 20 or 10 guinea pigs. Group 1 was 10 male and 10 female guinea pigs dosed with the highest concentration of LPT99, 797 μM, that was used in the dose range-finding substudy (Group 1). Group 2 was 5 male and 5 female animals that were not dosed with LPT99 (Group 2) during the induction phase and served as naïve controls; Group 2 was challenged with LPT99 on Day 28. LPT99 was topically administered to the skin in the shaved scapula area in a volume of 50 μL on Study Days 0, 7, and 14 (induction doses), and on Day 28 (challenge dose). These observations and measurements were performed: Clinical observations (daily), Body weights (weekly), Draize scoring (24 and 48 hours after bandage removal on Days 29 and 30), Primary Dermal Irritation Index (PDII) scoring of extent of irritation according to the scale in. The PDII (post-challenge) was calculated for the test article or vehicle by dividing the sum of the Total Irritation Score by the number of observations (e.g., 3 days×6 animals=18 (number of observations).

TABLE 5
Primary Dermal Irritation Index Scale

| Primary Dermal Irritation Value | Irritant Category |
|---|---|
| 0 | nonirritant |
| >0.0 to 0.5 | negligible irritant |
| >0.5 to 2.0 | mild irritant |
| >2.0 to 5.0 | moderate irritant |
| >5.0 to 7.0 | severe irritant |

No Draize scores were >0 at 24 or 48 hours after the challenge dose. The PDII score was 0, indicating that LPT99 was a nonirritant. No test-article-related changes were seen in mortality, clinical observations, body weights, or Draize scores.

Overall, topical dermal administration of 3 weekly induction doses and 1 challenge dose of 797 μM LPT99 was associated with no prevention-related effects. Based on the PDII score of 0, it was concluded that LPT99 was a nonirritant in this study.

### Example 13: Ototoxicity Study of LPT99 in Rats after Single TT Administration

A GLP-compliant auditory safety study of LPT99 solution was performed in rats after acute (single-dose) TT administration.

Materials and Methods

The study consisted of 10 groups of rats: 6 groups for toxicology (Groups 1 to 6, 20 males and 20 females per group), and 4 groups for toxicokinetics (Groups 7 to 10, 6 males and 6 females per group). A single bilateral TT administration (30 μL per ear) was performed on Day 0 with vehicle (Groups 1 and 10), 200 μM LPT99 (Groups 2 and 7), 400 μM LPT99 (Groups 3 and 8), 797 μM LPT99 (Groups 4 and 9); 400 mg/mL gentamicin (Group 5); and 0.9% Sodium Chloride, USP (Group 6).

All animals were assessed weekly for body weights and qualitative food consumption. In addition, otoscopic evaluation of the dosing sites (tympanic membrane, Groups 1 to 6) and ABR testing for hearing function were performed at predosing; and on Days 1, 7, and/or 14. Animals in the toxicology groups (Groups 1 to 6) were sacrificed on Day 1 (10 males and 10 females per group) and Day 14 (the remaining 10 males and 10 females per group). At necropsy, terminal blood samples were tested for hematology, serum chemistry, and coagulation parameters. Gross observations were recorded, and selected organs were weighed. One ear per animal in Groups 1 to 5 was collected and processed for cytocochleogram assessment.

Results

In this 14-day study, there were no LPT99- or gentamicin-related mortalities, clinical abnormalities, body weight changes, food intake abnormalities, or changes in clinical pathologies (hematology, serum chemistry, or coagulation). Otoscopic examination of the tympanic membranes (i.e., the study drug administration sites) revealed no statistically significant changes in erythema, edema, or wounds in the LPT99-prevented (200, 400, or 797 μM) groups compared with the vehicle control group on Days 1, 7, or 14. Gentamicin-related increases in otoscopic scores were seen on Days 1 (males had increased erythema and wound scores; females had increased wound scores) and 7 (females had increased erythema and edema scores). However, all otoscopic score increases had disappeared by Day 14.

Organ weight assessments at Day-1 or -14 necropsies revealed no statistically significant changes in absolute, weight-normalized, or brain-weight-normalized organ weights in any LPT99 group (dosed at 200, 400, or 797 μM); or in the gentamicin group compared with the respective controls on Day 1 or 14, with the exceptions described below.

On Day 1, the male rats in the middle dose group (400 μM) showed statistically significant (p<0.05) increases in weight-normalized liver weights compared with the vehicle controls. The increases appeared mild, with no correlation to the prevention doses; thus, the increases were considered toxicologically insignificant. On Day 14, males in the gentamicin control group had statistically significant (p≥0.05) increased weight-normalized heart weights compared with the saline controls.

Auditory brainstem response tests showed no reliable evidence of test-article-related hearing loss on Day 1 or 14. No statistically significant changes were seen in ABRs in the gentamicin group compared with the vehicle control group on Day 1 or 14, although a trend of hearing loss was observed in the gentamicin group on Day 1. Cytocochleogram analyses in cochlear samples collected at Days 1 and -14 necropsies showed no reliable evidence of test-article-related hair cell loss, except for one sample from a male in Group 4 (high dose group) sacrificed on Day 14; it is unclear whether this finding in one animal (1/20) was test-article related, since this animal exhibited no ABR alteration.

Taken together, a single TT dose of LPT99 at 200, 400, or 797 μM, administered in a volume of 30 μL per ear, was generally well tolerated.

The potential ototoxicity of LPT99 solution had been previously investigated in several non-GLP studies. An initial experiment with LPT99 solution in cyclodextrin at concentrations of 100 or 200 μM showed that LPT99, administered TT, did not produce a statistically significant increase in the thresholds in response to click or pure tones in the studied frequencies (8 to 40 kHz), or in functional parameters, including latencies and amplitudes of peaks ABR at Day 3 postprevention. In two additional non-GLP studies, TT administration of LPT99 solution in hydrogel (at 100, 300, 478, or 797 μM) did not produce a statistically significant increase in the thresholds in response to click or pure tones in the studied frequencies (8 to 40 kHz) at Days 3, 7, or 14 postprevention.

Summary of Examples

In vitro and in vivo experiments with LPT99 demonstrate that LPT99 as an Apaf1 inhibitor is capable of inducing a cytoprotective effect via inhibition of caspase activation.

Upon TT administration, LPT99 distributes locally to the cochlea and is not detected systemically and not considered to have systemic effects.

The in vivo efficacy of LPT99, tested in a rat model of CisPt-induced hearing loss, demonstrated that LPT99 administration has protective effects against CisPt-induced hearing loss.

The potential cardiotoxicity of LPT99-mediated effects on ion channels, including hERG potassium channels in CHO and human embryonic kidney (HEK) 293 cells, indicated LPT99 had a low cardiotoxic risk.

The neurological parameters in the acute toxicology study were unaffected by the prevention with LPT99 at the doses up to 750 mg/kg evaluated in the study.

LPT99 showed no evidence of genetic toxicity in the Ames test and is not considered to induce structural aberrations in cultured mammalian cells. The only impurity detected in LPT99 DS above ICH reporting thresholds (LPT102) is also considered non-mutagenic.

Topical dermal administration of LPT99 resulted in no prevention-related effects and was considered a nonirritant.

The potential ototoxicity of LPT99 administration was investigated and was generally well tolerated. Furthermore, LPT99 was found to not be phototoxic.

LPT99 administered via IP injection in Sprague-Dawley rats was generally safe and well tolerated at the doses up to 750 mg/kg evaluated in the study. There were no clinical signs observed in all the tested dose groups and no mortality was observed. Drug related changes were generally attributed to the acute inflammatory response around the injection site and were reversible.

The formulations were chemically and structurally stable for prolonged storage at room temperature.

We claim:

1. A sustained release formulation delivering an effective amount of a therapeutic or prophylactic agent for a period of at least three days for treatment of a condition, disease or disorder, the formulation comprising a solution of the agent in a synthetic polymer transitioning from a liquid state at room temperature, optionally in combination with a viscosity modifying agent and/or diluent, which can be injected through a 23 gauge needle to a gel state at body temperature.

2. The formulation of claim 1, wherein the synthetic polymer is a hydrophilic polymer.

3. The formulation of claim 1, wherein the synthetic polymer is an amphiphilic polymer.

4. The formulation of any one of claims 1-3, wherein the synthetic polymer is non-ionic.

5. The formulation of claim 4, wherein the synthetic polymer is a non-ionic, amphiphilic polymer.

6. The formulation of any one of claims 1-5, wherein the synthetic polymer is selected from the group consisting of synthetic polymers such as N-isopropylacrylamide (NiPAAM) polymers, poly(ethylene oxide)-b-poly(propylene oxide)-b-poly(ethylene oxide) (PEO-PPO-PEO), poly(ethylene glycol) (PEG)-biodegradable polyester copolymers, block copolymers of ethylene oxide and propylene oxide); and tetrafunctional block copolymer derived from sequential addition of propylene oxide and ethylene oxide to ethylenediamine.

7. The formulation of claim 1, wherein the synthetic polymer is a copolymer of ethylene oxide and propylene oxide.

8. The formulation of any one of claims 1-7, wherein the synthetic polymer is a tri-block copolymer composed of ethylene oxide and propylene oxide.

9. The formulation of any one of claims 1-8, wherein the synthetic polymer enhances solubility of the agent between at least about 30-fold, 50 fold, 100 fold, 200 fold, or 300 fold compared to a corresponding formulation lacking the synthetic polymer or to water.

10. The formulation of any one of claims 1-9, having a pH between about 6.8 and about 7.7, preferably 7.2.

11. The formulation of any one of claims 1-10, having an osmolality between about 240 mOsmol/kg and about 350 mOsmol/kg, preferably about 280 mOsmol/kg.

12. The formulation of any one of claims 1-11 wherein the agent is selected from the group consisting of anti-inflammatory agents, chemotherapeutic agents, antibiotic agents, anti-fungal agents, antiviral agents, corticosteroids, analgesics, immunomodulatory agents, local anaesthetics, aminoglycosides, compounds such as gentamicin for treatment of Menière's disease, neurotransmitters and neurotransmitter antagonists, growth factors, antioxidants, apoptosis inhibitors, nucleic acids, dyes, fluorophores, and other agents detectable by ultrasound, MRI, or x-ray.

13. A method of treating a condition, disease or disorder of the ear, comprising:
administering into the inner ear of a person in need or at risk of the condition, disease or disorder a sustained release formulation as a solution having dissolved therein an effective amount of therapeutic, prophylactic or diagnostic agent for a period of at least three days for treatment of the condition, disease or disorder, the formulation comprising a solution of the agent in a polymer transitioning from a liquid state at room temperature which can be injected through a 23 gauge needle to a stable hydrogel state at body temperature.

14. The method of claim 13 wherein the formulation is injected into a compartment of the ear.

15. The method of claim 14 wherein the formulation is administered trans-tympanically by injection of the formulation as a liquid.

16. A method of making a sustained release formulation delivering an effective amount of a therapeutic or prophylactic agent for a period of at least three days for treatment of a condition, disease or disorder, the formulation comprising a solution of the agent in a synthetic polymer transitioning from a liquid state at room temperature, optionally in combination with a viscosity modifying agent and/or diluent, which can be injected through a 23 gauge
a gel state at body temperature, comprising
dissolving the agent into the polymer formulation to form a uniform solution at room temperature or less.

17. The method of claim 16 wherein the drug has low solubility and is dissolved by application of mixing alone or with sonication.

18. The method of claim 16 or 17 wherein the formulation further comprises dispersants and viscosity modifiers enhancing the solubility of the agent in the formulation.

19. The method of any one of claims 16-18 wherein the pH is adjusted to pH 7.2

20. The method of any one of claims 16-18 further comprising lyophilizing the solution for rehydration prior to administration.
